(19) United States
(12) Patent Application Publication
(10) Pub. No.: US 2019/0297953 A1
(43) Pub. Date: Oct. 3, 2019

(54) LIQUID INJECTION PROTECTIVE MECHANISM, ATOMIZER AND ELECTRONIC CIGARETTE

(71) Applicant: Changzhou Patent Electronic Technology Co., LTD, Changzhou (CN)

(72) Inventors: Weihua QIU, Changzhou (CN); Wenbang WANG, Changzhou (CN)

(21) Appl. No.: 16/448,197

(22) Filed: Jun. 21, 2019

Related U.S. Application Data
(63) Continuation-in-part of application No. PCT/CN2017/114549, filed on Dec. 5, 2017.

(30) Foreign Application Priority Data
Dec. 21, 2016 (CN) 201621406359.X
Aug. 1, 2017 (CN) 201710647438.2

(51) Int. Cl. A24F 47/00; A61M 11/04

(52) U.S. Cl. CPC A24F 47/008; B65D 2215/04; A61M 11/042

(57) ABSTRACT

Provided is a liquid injection protective mechanism. It includes a cover, a connecting assembly, and a first locking assembly. The connecting assembly defines a liquid injection hole. The cover covers the liquid injection hole and is movable relative to the connecting assembly. The first locking assembly includes a locking hole defined on the cover, and a locking member capable of extending through the locking hole and detachably connected to the connecting assembly. When an end of the locking member extends through the locking hole and is connected to the connecting assembly, a movable stroke of the cover is restricted by the locking member, for preventing moving the cover to reveal the liquid injection hole. The liquid injection protective mechanism is therefore childproof. The present disclosure further provides an atomizer having the liquid injection protective mechanism and an electronic cigarette having the atomizer.

### CROSS-REFERENCE TO RELATED APPLICATIONS

The present application is a continuation-in-part of international application No. PCT/CN2017/114549 filed on Dec. 5, 2017, and claims priority to Chinese patent application Nos. 201621406359.X filed on Dec. 21, 2016, and 201710647438.2 filed on Aug. 1, 2017, and the entire disclosures of the foregoing applications are incorporated herein by reference.

### FIELD OF TECHNOLOGY

The invention relates to a technical field of smoking simulation, and more particularly, relates to a liquid injection protective mechanism, an atomizer and an electronic cigarette.

### BACKGROUND

Atomizer of a prior electronic cigarette includes a housing and a cover, the housing defines a liquid storage chamber therein, the cover covers an opening end of the liquid storage chamber to prevent leaking of the e-cigarette liquid from the liquid storage chamber. In general, in order to facilitate to conducting an e-cigarette liquid injection, the cover may be detachably connected to the housing. However, when children handle this type of electronic cigarette, the cover may be opened by an accident, and children may be exposed to and even swallow the e-cigarette liquid.

### SUMMARY

Accordingly, it is necessary to provide a liquid injection protective mechanism that is childproof and easy for liquid injection.

It is necessary to provide an atomizer having the liquid injection protective mechanism.

It is necessary to provide an electronic cigarette having the atomizer.

The technical solution adopted by the present disclosure to solve the problem is that:

A liquid injection protective mechanism, includes a cover, a connecting assembly, and a first locking assembly. The connecting assembly defines a liquid injection hole. The cover covers the liquid injection hole and is movable relative to the connecting assembly. The first locking assembly includes a locking hole defined on the cover, and a locking member capable of extending through the locking hole and detachably connected to the connecting assembly. When an end of the locking member extends through the locking hole and is connected to the connecting assembly, a movable stroke of the cover is restricted by the locking member, for preventing moving the cover to reveal the liquid injection hole.

Further, an outer diameter of a portion of the locking member matching the locking hole is equal to an inner diameter of locking hole, and/or an outer wall of the locking member is provided with a resisting edge, when the end of the locking member extends through the locking hole and is connected to the connecting assembly, the cover is restricted between the resisting edge and the connecting assembly.

Further, a sidewall of the locking hole is provided with a restricting protrusion, an external wall of the locking member defines a recessed restricting recessed groove, or, the sidewall of the locking hole defines a recessed restricting recessed groove, the external wall of the locking member is provided with a restricting protrusion, the restricting protrusion is movably embedded in the restricting recessed groove, for preventing a detachment of the locking member from the cover.

Further, the liquid injection protective mechanism further includes a movable ring, wherein the movable ring is rotatably sleeved on an outer of the connecting assembly, for preventing the connecting assembly to be dismantled.

Further, the locking member is a mouth piece.

Further, the connecting assembly includes a connecting base, the cover is detachably connected to the connecting base, or, the cover is movably positioned on the connecting base.

Further, an end of the cover is hinged to the connecting base, allowing the cover to be reversible relative to the connecting base.

Further, a sealing member is protruded from a lower surface of the cover and corresponds to the liquid injection hole, when the cover covers the connecting base, the sealing member is latched into the liquid injection hole.

Further, the cover is rotatably positioned on the connecting base, a rotation center of the cover is staggered from a central axial axis of the connecting base.

Further, the cover is slidably positioned on the connecting base, the connecting base is provided with a sliding rail, and the cover is provided with a sliding block engaging the sliding rail, or, the connecting base is provided with a sliding block, and the cover is provided with a sliding rail engaging the sliding block.

Further, the liquid injection protective mechanism further includes a second locking assembly, wherein the second locking assembly includes a first connecting portion positioned on the connecting assembly and a second connecting portion positioned on the cover, after a connection between the locking member and connecting assembly being removed, a movement of the cover is further required to separate the first connecting portion from the second connecting portion, for removing an engagement between the first connecting portion and the second connecting portion, and then the liquid injection hole can be opened.

Further, the connecting assembly defines a translational groove, the cover is provided with a rotation shaft, when the cover is moved, the rotation shaft slides along the translational groove, when the cover reaches a position permitting the first connecting portion to separate from the second connecting member, the cover is capable of rotating around the rotation shaft relative to the connecting assembly to reveal the liquid injection hole.

Further, the connecting assembly includes a connecting base and a pressing cover covering above the connecting base, the liquid injection hole is defined on the connecting base, the pressing cover defines a cutout with an opening at an end, the cutout is recessed from an edge of a side of the pressing cover, the liquid injection hole is received and revealed in the cutout.

Further, the cutout includes a connecting wall positioned on a side of the liquid injection hole, and two sidewalls respectively connected to opposite ends of the connecting wall, the two sidewalls extend toward a same direction, a surface of the connecting wall adjacent to the connecting base defines a recessed groove, the recessed groove is recessed from the connecting wall along a direction away from the cutout, the translational groove is defined at an end of each sidewall connecting with the connecting wall, the translation groove is recessed from a surface of the sidewall adjacent to the connecting base, the two surfaces of the two sidewalls defining the translational grooves are opposite to each other, the translational grooves are fluidly communicating with the cutout and the recessed groove.

Further, the cover includes a movable member, the rotation shaft is positioned on opposite sides of an end of the movable member, a covering portion is formed by extending outwardly from opposite sides of an end of the movable member opposite to the rotation shaft, the pressing cover defines a locking groove on a bottom of an end of the pressing cover away from the connecting wall, the locking groove is fluidly communicating with the cutout, the covering portion forms the second connecting portion, the locking groove forms the first connecting portion, when the movable member is moved to permit the covering portion latching into the locking groove, an end of the movable member adjacent to the rotation shaft is restricted in the recessed groove, when the movable member is moved to enable the covering portion to separate from the locking groove, the end of the movable member adjacent to the rotation shaft is moved out of the recessed groove, the movable member is reversible.

Further, the connecting assembly includes a lining member and the blocking member, the liquid injection hole is positioned on the lining member, the lining member further defines an embedding groove, the blocking member is embedded in the embedding groove, the translational groove is defined on an end of the blocking member and is arranged along a longitudinal direction of the blocking member, the cover includes a locking member, the rotation shaft is positioned on an end of the locking member.

Further, the connecting assembly further includes a snapping member positioned in the embedding groove and beneath an end of the blocking member away from the translational groove, the end of the blocking member away from the translational groove defines a pluggable hole, the snapping member and the pluggable hole cooperative constitute the first connecting portion, the locking member is provided with a claw at an end away from the rotation shaft, the claw forms the second connecting portion, when the locking member is moved to enable the claw to separate from the snapping member, and the claw is aligned to the pluggable hole, the locking member is reversible.

Further, the claw defines a latching groove, the snapping member is provided with a protrusion corresponding to the latching groove, when the first connecting portion engages the second connecting portion, the protrusion latches into the latching groove, or, both the snapping member and the claw are provided with a magnetic member, when the first connecting portion engages the second connecting portion, the snapping member and claw are absorbed by the magnetic members.

Further, the connecting assembly further includes an elastic member, when the locking member is moved, the elastic member resists an end of the locking member provided with the rotation shaft all the time.

An atomizer, includes any one of aforementioned liquid injection protective mechanism.

Further, the atomizer further includes a housing, a base seat assembly, and an atomizing head, wherein the connecting assembly is positioned on an end of the housing, the base seat assembly is positioned on an end of the housing opposite to the connecting assembly, the housing forms a liquid storage chamber fluidly communicating with the liquid injection hole, the atomizing head is received in the liquid storage chamber.

An electronic cigarette, includes any one of aforementioned atomizers.

The advantages of the present disclosure are described as follows:

In the liquid injection protective mechanism provided by the present disclosure, when an end of the locking member extends through the locking hole and is connected to the connecting assembly, the movable stroke of the cover is restricted by the locking member, for preventing moving the cover to reveal the liquid injection hole. When a liquid injection is required, the locking member is required to be unlocked firstly, and then the cover is moved to reveal the liquid injection hole. Because the liquid injection hole can only be opened by a complicated operation procedure, thus children cannot open the liquid injection hole causally, and exposure or swallowing of the e-cigarette liquid by children can be avoided, so that it is childproof. The present disclosure further provides an atomizer having the liquid injection protective mechanism and an electronic cigarette having the atomizer.

### BRIEF DESCRIPTION OF THE DRAWINGS

The invention is specifically illustrated with reference to accompanying drawings and embodiments in the following description.

FIG. 1 is a structural view of an atomizer of a first embodiment of the present disclosure (a cover is positioned on a reversed state);

FIG. 2 is a partially and exploded view of the atomizer of FIG. 1;

FIG. 3 is an exploded view of a liquid injection protective mechanism of the atomizer of FIG. 1 (a mouth piece is not shown);

FIG. 4 is an exploded view of the liquid injection protective mechanism of the atomizer of FIG. 3, viewed from another aspect;

FIG. 5 is a structural view of a lining member of the liquid injection protective mechanism of the atomizer of FIG. 3;

FIG. 6 is a cross-sectional view of the liquid injection protective mechanism of the atomizer of FIG. 3 (the cover is positioned on a locking state);

FIG. 7 is another cross-sectional view of the liquid injection protective mechanism of the atomizer of FIG. 3 (the cover is positioned on an unlocking state);

FIG. 8 is a cross-sectional view of the atomizer of FIG. 1 (the mouth piece is not shown);

FIG. 9 is an enlarged view of circled portion A of the atomizer of FIG. 2;

FIG. 10 is a structural view of an atomizer of a second embodiment of the present disclosure (a cover is positioned on a reversed state);

FIG. 11 is a partially and exploded view of the atomizer of FIG. 10;

FIG. 12 is an exploded view of a liquid injection protective mechanism of the atomizer of FIG. 10 (a mouth piece is not shown);

FIG. 13 is a cross-sectional view of the liquid injection protective mechanism of the atomizer of FIG. 12 (the cover is positioned on a reversed state);

FIG. 14 is a cross-sectional view of the atomizer of FIG. 10;

FIG. 15 is a structural view of an atomizer of a third embodiment of the present disclosure (a cover is positioned on a locking state);

FIG. 16 is a cross-sectional view of the atomizer of FIG. 15;

FIG. 17 is a schematic view of the atomizer of FIG. 15, showing another state (the cover is positioned on an unlocking state);

FIG. 18 is a schematic view of the atomizer of FIG. 15, showing a further state (a liquid injection hole is revealed);

FIG. 19 is a structural view of an atomizer of a fourth embodiment of the present disclosure (a cover is positioned on a locking state);

FIG. 20 is a cross-sectional view of the liquid injection protective mechanism of the atomizer of FIG. 19;

FIG. 21 is a schematic view of the atomizer of FIG. 19, showing another state (the cover is positioned on an unlocking state);

FIG. 22 is a schematic view of the atomizer of FIG. 19, showing a further state (a liquid injection hole is revealed);

FIG. 23 is a structural view of an atomizer of a fifth embodiment of the present disclosure (a cover is positioned on a locking state);

FIG. 24 is a cross-sectional view of the liquid injection protective mechanism of the atomizer of FIG. 23;

FIG. 25 is a schematic view of the atomizer of FIG. 23, showing another state (the cover is positioned on an unlocking state);

FIG. 26 is a schematic view of the atomizer of FIG. 23, showing a further state (a liquid injection hole is revealed);

FIG. 27 is a structural view of an atomizer of a sixth embodiment of the present disclosure (a cover is positioned on a locking state);

FIG. 28 is a cross-sectional view of the liquid injection protective mechanism of the atomizer of FIG. 27;

FIG. 29 is a schematic view of the atomizer of FIG. 27, showing another state (the cover is positioned on an unlocking state);

FIG. 30 is a schematic view of the atomizer of FIG. 27, showing a further state (a liquid injection hole is revealed).

Designations and reference numerals of the part and component in the accompanying drawings.

| | | |
|---|---|---|
| atomizer 100, 200, 300, | | housing 10 |
| 400, 500, 600 | | |
| locking groove 4163 | liquid storage chamber 101 | atomizing head 20 |
| atomizing sleeve 21 | second electrode 22 | atomizing chamber 201 |
| liquid intake hole 202 | base seat assembly 30 | base seat 31 |
| first electrode 32 | electrode sleeve 33 | air-regulating ring 34 |
| connecting tube 424, 311 | first insulating member 35 | second insulating member 36 |
| connecting assembly 40 | aerosol exhaust hole 401 | liquid injection hole 402 |
| connecting base 411 | lining member 414 | blocking member 415 |
| connecting post 4111 | perforation 4142, 4241 | embedding groove 4144 |
| first convex boss 4145 | second convex boss 4146 | slot 4147 |
| snapping member 420 | protrusion 4181 | aligning groove 4151 |
| insertion hole 4152 | chamber 4153 | pluggable hole 4154 |
| restricting groove 4155 | translational groove | elastic member 419 |
| | 4156, 4165 | |
| clamping portion 4191 | resisting portion 4192 | pressing cover 416 |
| sealing ring 418 | cutout 4161 | connecting wall 4162 |
| sidewall 4164 | recessed groove 4166 | covering portion 432 |
| movable member 433 | sealing groove 4143 | pin 417 |
| ventilation member 42 | restricting recessed groove 601 | restricting portion 5011 |
| first fixing member 431 | second fixing member 435 | mouth piece 60 |
| aerosol exhaust passage 423 | sliding member 46 | cover 50 |
| through hole 501 | locking member 434 | inserting portion 4331 |
| aligning hole 4341 | sealing pad 45 | first connecting member |
| | | 4342 |
| claw 4343 | latching groove 4344 | second connecting portion |
| | | 421 |
| first connecting portion 422 | second connecting member | resisting edge 602 |
| | 4345 | |
| rotation shaft hole 4347 | rotation shaft 436, 503 | movable ring 403 |
| sealing member 502 | rotating member 44 | guiding groove 4149 |

### DETAILED DESCRIPTION OF EMBODIMENTS

Embodiments of the disclosure are described more fully hereinafter with reference to the accompanying drawings. The various embodiments of the disclosure may, however, be embodied in many different forms and should not be construed as limited to the embodiments set forth herein. Rather, these embodiments are provided so that this disclosure will be thorough and complete, and will fully convey the scope of the disclosure to those skilled in the art.

It should be noted that when an element is described as “fastened to” or “disposed on” another element, the element may be directly on the another element, or a mediate element may also exist; when an element is described as “connected to” another element, the element may be directly connected to the another element, or a mediate element may also exist.

Unless otherwise defined, all terms (including technical and scientific terms) used herein have the same meaning as commonly understood by one of ordinary skill in the art to which this disclosure belongs. The terms used herein in the specification of the present disclosure are for the purpose of describing the embodiments only and are not intended to limit the invention. As used herein, the term “and/or” includes any and all combinations of one or more of the associated listed items.

### The First Embodiment

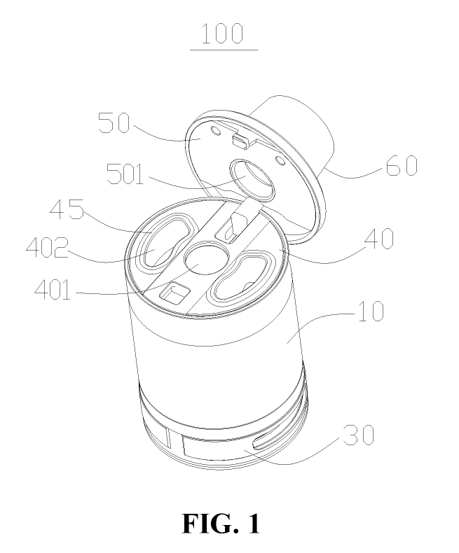

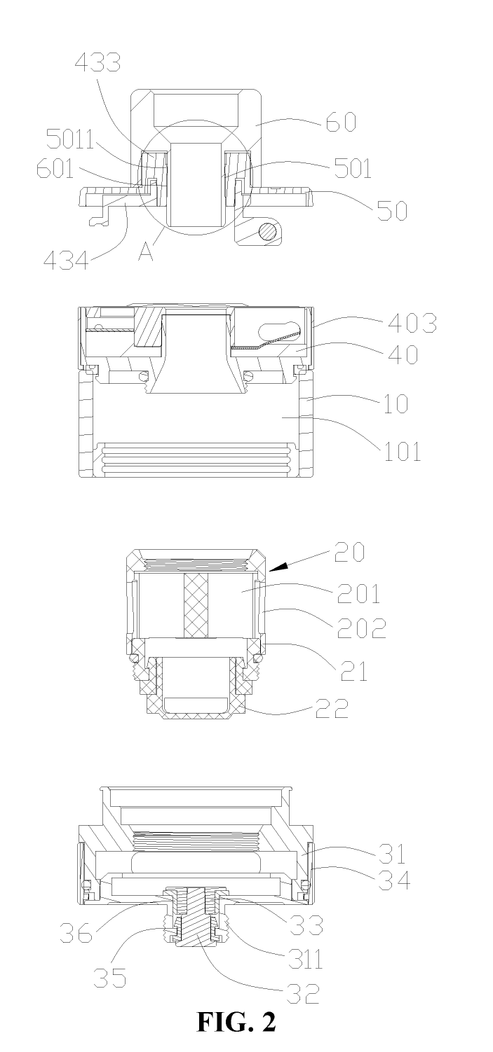

Referring to FIG. 1 and FIG. 2, an electronic cigarette is provided by the first embodiment of the present disclosure, the electronic cigarette includes an atomizer 100 and a power source mechanism (not shown) electrically connected to the atomizer 100. Wherein, the atomizer 100 includes a housing 10, an atomizing head 20 received in the housing 10, a base seat assembly 30 connected to an end of the housing 10, a connecting assembly 40 connected to an end of the housing 10 opposite to the base seat assembly 30, and a cover 50 covering the connecting assembly 40, the power source mechanism is detachably mounted on the base seat assembly 30 and is electrically connected to the atomizing head 20. The housing 10 stores e-cigarette liquid therein, and can supply the e-cigarette liquid to the atomizing head 20. The atomizing head 20 is driven by the power source mechanism to heat the e-cigarette liquid to generate aerosol for user to inhale.

The housing 10 has a substantially hollow cylindrical structure having two openings at opposite ends. The housing 10 is provided with a liquid storage chamber 101 configured to storage the e-cigarette liquid. In the illustrated embodiment, the interior chamber of the housing 10 is the liquid storage chamber 101. The housing 10 is made of transparent or translucent materials, allowing user to observe an amount of the e-cigarette liquid in the liquid storage chamber 101 via the housing 10, facilitating user to perform an e-cigarette liquid injection timely. In the illustrated embodiment, the housing 10 is made of glass. It can be understood that, in alternative embodiment not shown, the housing 10 can be further sleeved with a protective sleeve made of rigid materials such as stainless steel, for protecting the housing 10.

The base seat assembly 30 includes a base seat 31 mounted on a lower end of the housing 10, a first electrode 32 mounted on the base seat 31, an electrode sleeve 33 and an air-regulating ring 34 rotatably sleeved on an outer of the base seat 31.

Referring to FIG. 2 again, the base seat 31 has a substantially hollow cylindrical structure with an opening at an upper end thereof. The base seat 31 forms a connecting tube 311 fluidly communicating with an interior chamber of the base seat 31, the connecting tube 311 extends downwardly from a center of a bottom of the base seat 31. The first electrode 32 is mounted within the connecting tube 311, the electrode sleeve 33 is sleeved on the upper end of the first electrode 32, and is electrically connected to the atomizing head 20. Specifically, the power source mechanism is connected to the connecting tube 311 of the base seat 31 via threads, enabling the first electrode 32 to be electrically connected to the power source mechanism, thereby enabling the power source mechanism to provide power for the atomizing head 20. In the illustrated embodiment, a first insulating member 35 is clamped between the first electrode 32 and the connecting tube 311, a second insulating member 36 is clamped between the electrode sleeve 33 and the connecting tube 311, thereby realizing a power isolating function. The first insulating member 35 and the second insulating member 36 are made of adiabatic materials. It can be understood that, the first insulating member 35 and the second insulating member 36 includes, but not limited to silica gel or rubber.

The air-regulating ring 34 has a substantially cylindrical structure with two ends cutting through each other. The circumferential wall of the base seat 31 defines an air intake hole (not shown) in fluid communication with the interior chamber of the base seat 31. The circumferential wall of the air-regulating ring 34 defines an air-regulating hole (not shown) corresponding to the air intake hole and fluidly communicating with outside. When the air-regulating ring 34 is rotated, a communicating area between the air-regulating hole and the air intake hole can be adjusted, facilitating for the user to adjust the intake air flow according to a requirement.

The atomizing head 20 includes an atomizing sleeve 21 received in the liquid storage chamber 101, a heating assembly (not shown) received in the atomizing sleeve 21, and a second electrode 22 mounted on a bottom of the atomizing sleeve 21. Wherein, the upper end and the lower end of the atomizing sleeve 21 fluidly communicate with each other, the atomizing sleeve 21 forms an atomizing chamber 201 therein, the atomizing sleeve 21 defines a liquid intake hole 202 fluidly communicating the liquid storage chamber 101 with the atomizing chamber 201. The heating assembly is positioned within the atomizing chamber 201. The heating assembly includes a heating member (not shown) and a liquid guiding member (not shown) which contact each other, the liquid guiding member is configured to absorb the e-cigarette liquid in the liquid storage chamber 101 via the liquid intake hole 202, the heating member is configured to heat the e-cigarette liquid on the liquid guiding member, causing the e-cigarette liquid to generate aerosol. Wherein, the heating member can be a heating wick, the liquid guiding member can be a liquid guiding cotton. With regard to the setting mode between the heating member and the liquid guiding member, the liquid guiding cotton can wrap the heating wick, or the heating wick can wind the liquid guiding cotton. Further, the lower end of the second electrode 22 contacts and is electrically connected to the electrode sleeve 33, the upper end of the second electrode 22 is electrically connected to the heating member, thereby enabling the power source mechanism to provide power for the heating member.

Referring to FIG. 1 again, the connecting assembly 40 is assembled to an upper end of the housing 10, the cover 50 can move between a locking position and an unlocking position relative to the connecting assembly 40. The connecting assembly 40 includes an aerosol exhaust hole 401 fluidly communicating with the atomizing chamber 201 and a liquid injection hole 402 fluidly communicating with the liquid storage chamber 101. The cover 50 defines a through hole 501. Wherein, when the cover 50 is positioned on the locking position relative to the connecting assembly 40, the cover 50 covers the connecting assembly 40 and cannot be reversed relative to the connecting assembly 40. The through hole 501 is fluidly communicating with the aerosol exhaust hole 401, the liquid injection hole 402 is sealed. When the cover 50 is moved to the unlocking position from the locking position relative to the connecting assembly 40 via a translational pushing by an external force, the cover 50 can be reversed relative to the connecting assembly 40 and is opened, the liquid injection hole 402 is revealed.

Therefore, when the electronic cigarette is used, the though hole 501 of the cover 50 is fluidly communicating with the aerosol exhaust hole 401, facilitating for the aerosol atomized and generated in the atomizing chamber 201 flows out via the through hole 501, for user to inhale. At the time, the liquid injection hole 402 is sealed, and a liquid injection operation cannot be performed. When a liquid injection operation is required, the cover 50 is moved to the unlocking position from the locking position via a translational pushing, and is reversed relative to the connecting assembly 40 to be opened, allowing the liquid injection hole 402 to be revealed, user can performs a liquid injection operation.

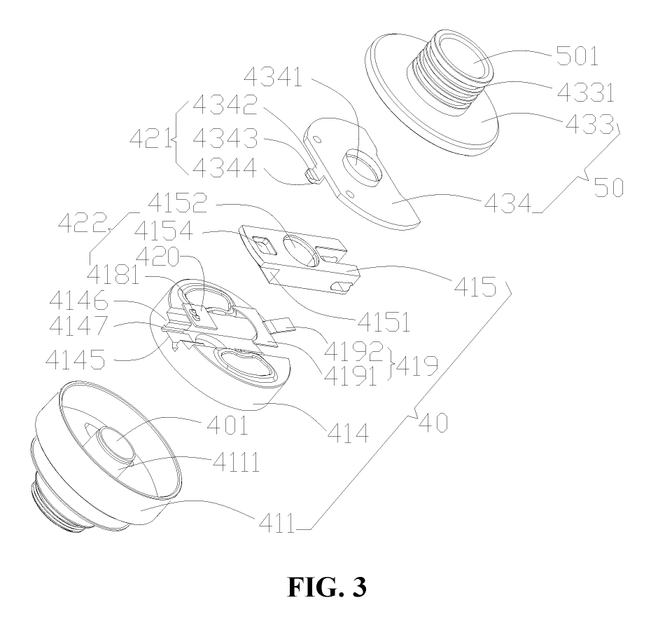

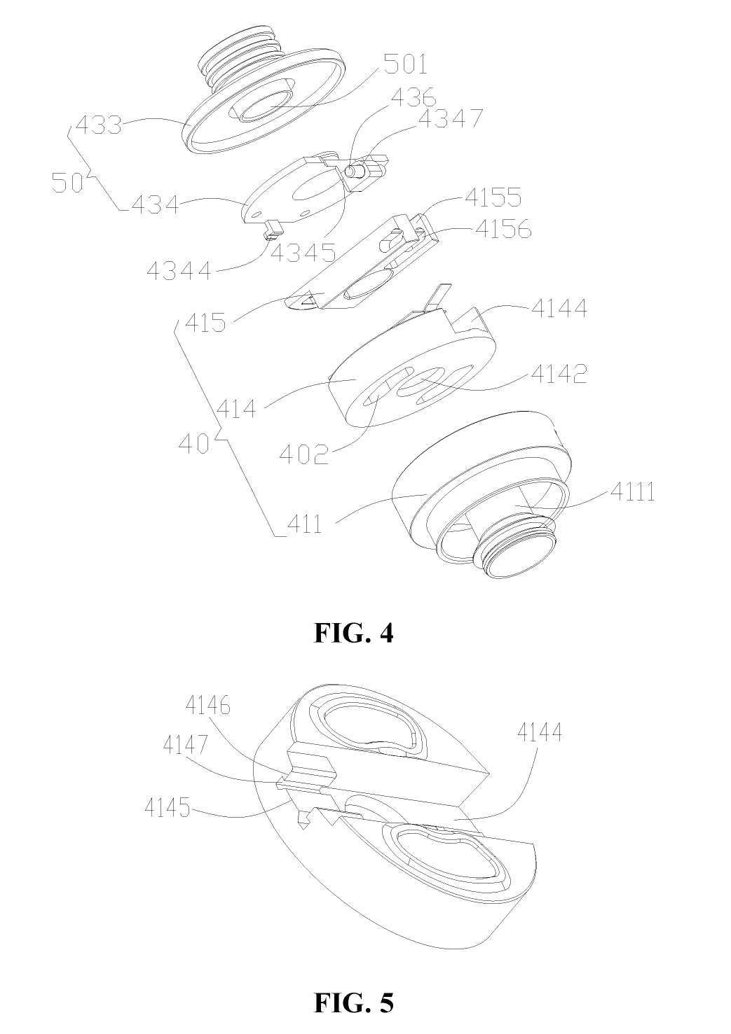

Specifically, referring to FIG. 3 and FIG. 4 at the same time, in the illustrated embodiment, the connecting assembly 40 includes a connecting base 411, a lining member 414 received in the connecting base 411, and a blocking member 415 embedded in the lining member 414.

The connecting base 411 has a substantially hollow cylindrical structure with an opening at an upper end. The connecting base 411 is positioned on an end of the housing 10 away from the base seat assembly 30. The connecting base 411 is provided with a connecting post 4111 on a bottom surface, the connecting post 4111 is hollow and cuts through the bottom surface of the connecting base 411. An end of the connecting post 4111 located below the bottom surface of the connecting base 411 is connected to the atomizing sleeve 21 via threads, for fixing the connecting assembly 40 to the atomizing head 20 and permits a ventilation of the aerosol in the atomizing chamber 201. An end of the connecting post 4111 away from the atomizing head 20 is received in the connecting base 411, the aerosol exhaust hole 401 is formed by a port of the end of the connecting post 4111 away from the atomizing head 20.

The lining member 414 has a substantially cylindrical structure, the lining member 414 defines a perforation 4142 at a center, the perforation 41042 extends through opposite end surfaces of the lining member 414, the lining member 414 is received in the connecting base 411. When the lining member 414 is received in the connecting base 411, an end of the connecting post 4111 received in the connecting base 411 extends through the perforation 4142. The lining member 414 defines an embedding groove 4144 from a surface of the lining member 414 facing the cover 50. The embedding groove 4144 is arranged along a radial direction of the lining member 414. The embedding groove 4144 is configured to latch with a blocking member 415, for fixing the blocking member 415. The embedding groove 4144 has a substantially elongated structure, opposite ends of the embedding groove 4144 cut through the circumferential wall of the lining member 414. A width of the embedding groove 4144 is greater than an external diameter of the perforation 4142, the embedding groove 4144 is fluidly communicating with the perforation 4142. Referring to FIG. 5, a bottom wall of an end of the embedding groove 4144 is provided with a first convex boss 4145, the embedding groove 4144 is provided with two second convex bosses 4146 on opposite sidewalls adjacent to the first convex boss 4145. Each of the two second convex bosses 4146 defines a slot 4147 at a bottom, each slot 4147 cuts through two sidewalls of the corresponding second convex boss 4146, the two sidewalls are arranged along a longitudinal direction of the embedding groove 4144. An insertion groove (not labeled) is constituted by a space formed between the top surface of the first convex boss 4145 and the upper wall of the slot 4147.

In the illustrated embodiment, a number of the liquid injection holes 402 is two, the two liquid injection holes 402 are positioned on opposite sides of the embedding groove 4144. The liquid injection hole 402 cuts through opposite ends of the lining member 414. It can be understood that, in alternative embodiments not shown, the number of liquid injection hole 402 is at least one, the positions of the liquid injection hole 402 is not limited, merely the liquid injection can be accomplished. In the illustrated embodiment, the connecting base 411 defines a via hole (not shown) fluidly communicating with the liquid injection hole 402. The liquid injection hole 402 is fluidly communicating with the liquid storage chamber 101 via the via hole.

The connecting assembly 40 further includes a snapping member 420, the snapping member 420 has a substantially sheet structure, and tightly engages the insertion groove. When the snapping member 420 is assembled, the snapping member 420 is merely plugged into the insertion groove along the opening end of the insertion grove. In the illustrated embodiment, the snapping member 420 is provided with two protrusions 4181 protruding from a surface of the snapping member 420 facing the cover 50. The two protrusions 4181 are made of elastic materials, when the protrusion 4181 is exerted by an external force, it can be deformed, when the external force is removed, the protrusion 4181 can return to an original position.

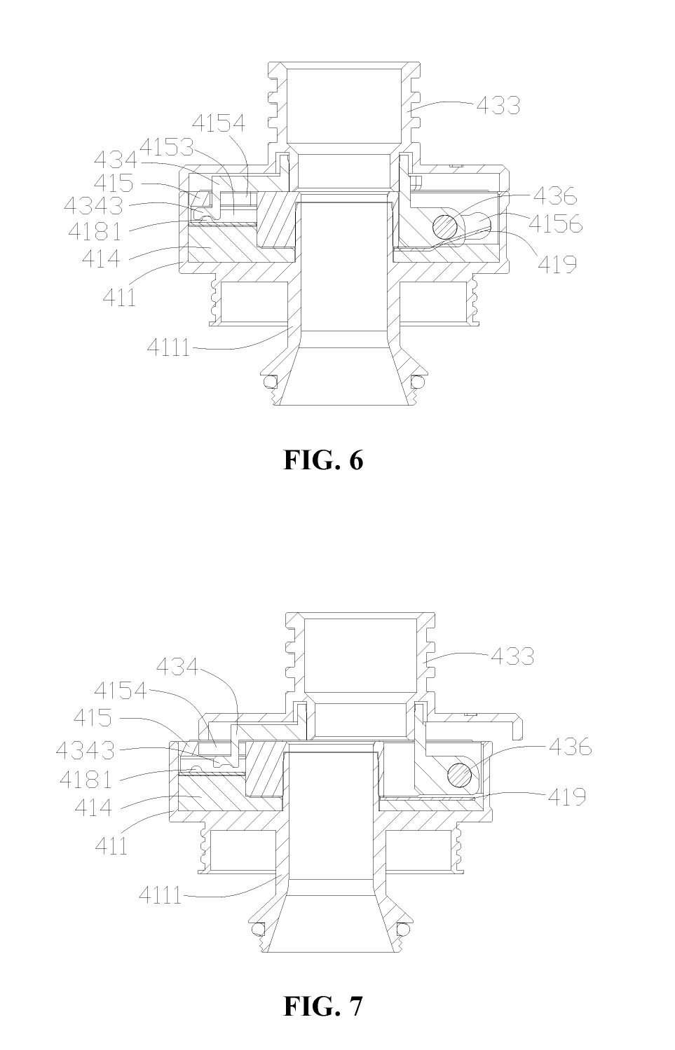

The blocking member 415 has a substantially elongated shape, the blocking member 4151 defines an aligning groove 415 corresponding to the second protrusion 4181, the aligning groove 4151 is recessed from the surface of the blocking member 415 away from the cover 50. The blocking member 415 defines an insertion hole 4152 corresponding to the connecting post 4111. The insertion hole 4152 cuts through the upper surface and the lower surface of the blocking member 415. Referring to FIG. 6, when the blocking member 415 is embedded in the embedding groove 4144, the connecting post 4111 is plugged into the insertion hole 4152, a bottom wall of the aligning groove 4151 resists the second convex boss 4146, thereby, a chamber 4153 is formed between the blocking member 415 and the snapping member 420. It is obviously that, the chamber 4153 is a part of the aligning groove 4151. In the illustrated embodiment, the blocking member 415 defines a pluggable hole 4154 on a surface of the blocking member 415 facing the cover 50, the pluggable hole 4154 is fluidly communicating with the chamber 4153. It can be understood that, the pluggable hole 4154, the snapping member 420 and the protrusion 4181 cooperatively constitute a first connecting portion 422.

The blocking member 415 defines a restricting groove 4155 at an end opposite to the pluggable hole 4154, the restricting groove 4155 is arranged along a longitudinal direction of the blocking member 415, and cuts through the upper surface and the lower surface of the blocking member 415. Opposite sidewalls of the restricting groove 4155 defines translational grooves 4156 along a longitudinal direction of the blocking member 415, the translational groove 4156 has a substantially kidney shape.

In the illustrated embodiment, the connecting assembly 40 further includes an elastic member 419 clamped between the lining member 414 and the blocking member 415. The elastic member 419 includes a clamping portion 4191 and a resisting portion 4192 connected to the clamping portion 4191. The clamping portion 4191 has a substantially square shape, the resisting portion 4192 has a substantially elongated structure, the clamping member 4191 is clamped between the lining member 414 and the blocking member 415. The resisting portion 4192 is connected to a side of the clamping portion 4191 away from the insertion hole 4152, and is arranged in an inclined manner along a direction away from the bottom wall of the embedding groove 4144. The resisting portion 4192 processes elasticity, and presses the resisting portion 4192 downwardly, an end of the clamping member 4191 away from the resisting portion 4192 can move downwardly, when the pressure is removed, the resisting portion 4192 can return to an original position. In the illustrated embodiment, the clamping portion 4191 and the resisting portion 4192 can be a stainless steel elastic sheet, which is integrally formed. It can be understood that elastic part such as spring can also be applied.

Referring to FIG. 3 and FIG. 4 again, the cover 50 includes a movable member 433 and a locking member 434 positioned beneath the movable member 433.

Specifically, the movable member 433 has a substantially disc structure, the through hole 501 is defined on the center of the movable member 433 and the sidewall of the through hole 501 forms an inserting portion 4331 extending along a direction away from the connecting assembly 40.

The locking member 434 has a substantially kidney and planar shape. The locking member 434 is fixedly assembled to the surface of the movable member 433 away from the inserting portion 4331, when the cover 50 is positioned on the locking position, the locking member 434 can seal and block the liquid injection hole 402, thereby sealing the liquid injection hole 402. The locking member 434 defines an aligning hole 4341 corresponding to the through hole 501, when the locking member 434 is connected to the movable member 433, the aligning hole 4341 is fluidly communicating with the through hole 501. In the embodiment, the locking member 434 is plugged in the movable member 433. It can be understood that, in alternative embodiment not shown, the locking member 434 can be connected to the movable member 433 via latching or adhering, or the locking member 434 and the movable member 433 are integrally formed, which is not limited herein.

Referring to FIG. 1 again, in order to improve an air-tightness of the liquid injection hole 402 applied by the locking member 434, a sealing pad 45 is positioned on a periphery of the liquid injection hole 402.

The locking member 434 is provided with a first connecting member 4342 protruding downwardly from the surface of the locking member 434 opposite to the movable member 433, the first connecting member 4342 is adjacent to the pluggable hole 4154 of the blocking member 415. The first connecting member 4342 forms a claw 4343 extending outward from an end away from the locking member 434. The claw 4343 defines a latching groove 4344 recessed from a surface opposite to the locking member 434, the latching groove 4344 engages the protrusion 4181. The claw 4343 can be plugged into the chamber 4153 via the pluggable hole 4154. When the locking member 434 performs a translational movement, the claw 4343 can be driven by the locking member 434 to move in the chamber 4153, thereby enabling the protrusion 4181 to latch into the latching groove 4344, or enabling the protrusion 4181 to be separated from the latching groove 4344. It can be understood that, the first connecting member 4342, the claw 4343, and the latching groove 4344 cooperative constitute a second connecting portion 421. When the cover 50 is positioned on the locking position, the first connecting portion 422 latches with the second connecting portion 421. At the time, the protrusion 4181 is latched into the latching groove 4344, the periphery of the pluggable hole 4154 resists the claw 4343, thereby restricting a reversing action of the locking member 434 relative to the connecting assembly 40. In the illustrated embodiment, the protrusion 4181 is made of elastic materials, the protrusion 4181 is deformed and latched into the latching groove 4344. It can be understood that, in alternative embodiment not shown, the claw 4343 is made of elastic material, the claw 4343 is deformed to enable the protrusion 4181 to be latched into the latching groove 4344.

Obviously, it can be understood that, in alternative embodiment not shown, the first connecting portion 422 and the second connecting portion 421 are provided with magnetic members such magnets, when the cover 50 is positioned on the locking mechanism, the first connecting portion 422 and the second connecting portion 421 are magnetically connected. Specifically, the second connecting portion 421 includes a claw 4343, the claw 4343 is a magnetic member or is provided with a magnetic member. The first connecting portion 422 includes a snapping member 420 and a protrusion 4181. The protrusion 4181 is provided with magnetic member such as magnet, when the claw 4343 move in place, the magnetic member of the claw 4343 absorbs the magnetic member of the protrusion 4181, thereby locking the locking member 434, improving an air-tightness between the cover 50 and the connecting assembly 40. When the cover 50 is positioned on the unlocking position, the first connecting portion 422 is detached from the second connecting portion 421, the periphery of the pluggable hole 4154 does not resist the claw 4343 anymore, thereby allowing the cover 50 to be reversed. It can be understood that, in alternative embodiment, each of the first connecting portion 422 and the second connecting portion 421 can adopt other shape, structure or connecting mode. For example, each of the first connecting portion 422 and the second connecting portion 421 can include claws, the claw of the second connecting portion 421 can have a shape same as that in the illustrated embodiment, the claw of the first connecting member 4222 is same as the claw 4343 of second connecting portion 421 in an inversed state, such that the claws can latch with each other.

It can be understood that, the first connecting portion 422 and the second connecting portion 421 cooperative constitute a second locking assembly. When the cover 50 is moved to allow the first connecting portion 422 to engage the second connecting portion 421, the cover 50 is locked, when the cover 50 is moved to allow the first connecting portion 422 to separate from the second connecting portion 421, the cover 50 is unlocked.

The locking member 434 is provided with a second connecting member 4345 protruding from a surface opposite to the movable member 433, the second connecting member 4345 is adjacent to the restricting groove 4155 of the blocking member 451. The second connecting member 4345 has an “L” structure, the second connecting member 4345 engages the restricting groove 4155 and can slide within the restricting groove 4155, the second connecting member 4345 defines a rotation shaft hole 4347 on a sidewall, the sidewall is located on an end of the second connecting member 4345 extending along a sliding direction. In the illustrated embodiment, the cover 50 further includes a rotation shaft 436, the rotation shaft 436 is mounted in the rotation shaft hole 4347 and is slidably embedded in the translational groove 4156. When the rotation shaft 436 reaches an end of the translational groove 4156 away from the pluggable hole 4154, and then the cover 50 is reversed, the cover 50 is allowed to rotate around the connecting assembly 40 and take the rotation shaft 436 as a rotation center. It should be noted that, the rotation shaft 436 is located at an arbitrary position in the translational groove 4156, the resisting portion 4194 of the elastic member 419 elastically resists the second connecting member 4345.

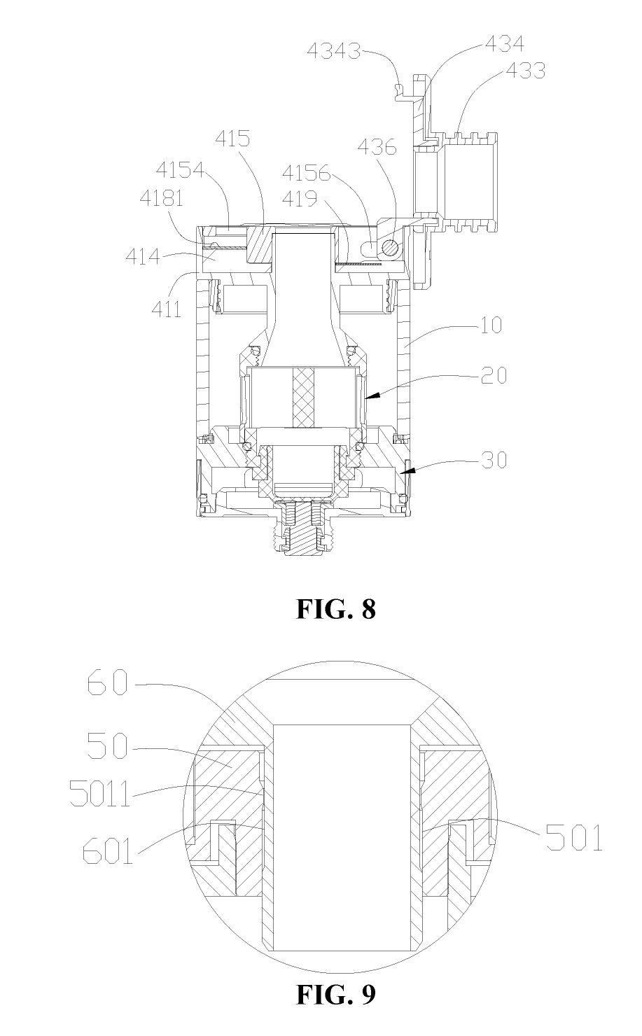

Referring to FIG. 7 and FIG. 8, when user want to perform a liquid injection, the cover 50 is firstly moved away from the pluggable hole 4154 via a translational pushing, enabling the locking member 434 to be driven by the movable member 433 to move, the first connecting portion 422 and the second connecting portion 421 are unlocked. For example, the latching groove 4344 of the claw 4334 is detached from the protrusion 4181. When the claw 4343 is aligned to the pluggable hole 4154, at the time, the rotation shaft 436 reaches an end of the translational groove 4156 away from the pluggable hole 4154, the cover 50 is positioned on the unlocking position. Then the cover 50 is reversed to reveal the liquid injection hole 402, the user can thereby perform a liquid injection operation. Referring to FIG. 6, when the liquid injection is accomplished and the liquid injection hole 402 is required to be closed, the cover 50 is reversed first, causing the claw 4343 to be received in the chamber 4153 via the pluggable hole 4154, at the time, the cover 50 is positioned on the unlocking portion. The cover 50 is moved toward the protrusion 4181 via a translational pushing, enabling the latching groove 4344 to latch with the protrusion 4181, the periphery of the pluggable hole 4154 resists the claw 4343, thereby preventing a reversing action of the locking member 43 relative to the connecting assembly 40, i.e., the cover 50 cannot be reversed relative to the connecting assembly 40. At the time, the cover 50 is positioned on the locking position, the first connecting portion 422 and the second connecting portion 421 are locked, the cover 50 covers the connecting assembly 40, the liquid injecting hole 402 is sealed. The user can perform a smoking, and cannot conduct a liquid injection operation.

It should be noted that, in the illustrated embodiment, when the cover 50 is positioned on the locking position, on one hand, the elastic member 419 has a resisting action to the second connecting member 4345, one the other hand, a latching action is existed between the latching groove 4344 and the protrusion 4181, thereby permitting the cover 50 to remain on the locking position all the time. The cover 50 cannot slide to the unlocking position casually due to a weak locking force. It can be understood that, in alternative embodiment not shown, the latching structure of the latching groove 4344 and the protrusion 4181 or the elastic member 419 can be substituted, a casual sliding of cover 50 can also be avoided.

In the illustrated embodiment, the atomizer 100 further includes a first locking assembly. It can be understood that, the connecting assembly 40, the cover 50, the first locking assembly, and the second locking assembly cooperatively constitute the liquid injection protective mechanism. The first locking assembly includes a locking hole positioned on the cover 50, and a locking member extending through the locking hole and detachably connected to the connecting assembly 40. When an end of the locking member extends through the locking hole and is connected to the connecting assembly 40, the movable stroke of the cover 50 is restricted by the locking member, therefore, the cover 50 cannot move to reveal the liquid injection hole 402.

Referring to FIG. 2 and FIG. 9 again, in the illustrated embodiment, the locking hole is a through hole 501 defined on the movable member 433 of the cover 50, the locking member is a mouth piece 60 extending through the through hole 501 and connected to the connecting assembly 40. Specifically, the mouth piece 60 has a substantially hollow cylindrical structure having two openings at opposite ends, an end of the mouth piece 60 extends through the through hole 501 and is then plugged into the insertion hole 4152 of the blocking member 415. In the illustrated embodiment, the sidewall of the through hole 501 is provided with a restricting protrusion 5011, the external circumferential wall of the mouth piece 60 defines a restricting recessed groove 601 recessed from the mouth piece 60, the restricting protrusion 5011 is movably embedded in the restricting recessed groove 601, when the mouth piece 60 is separated from the cover 50, the mouth piece 60 cannot lose. Specifically, when the mouth piece 60 is drawn outwardly to enable the restricting protrusion 5011 to resist an end of the restricting recessed groove 601, an end of the mouth piece 60 is separated from the insertion hole 4152, and is received in the through hole 501. At the time, a locking relationship between the connecting assembly 40 and the cover 50 is removed, thus the cover 50 can be pushed relative the connecting assembly 40, and the cover 50 is then reversed to reveal the liquid injection hole 402, user can conduct a liquid injection operation. When the mouth piece 60 is plugged inwardly to enable the restricting protrusion 5011 to resist an opposite end of the restricting recessed groove 601, the mouth piece 60 is plugged into the insertion hole 4152, thereby locking the connecting assembly 40 and the cover 50, user can smoke via the mouth piece 60, but cannot perform a liquid injection operation. It can be understood that, in alternative embodiment not shown, the restricting protrusion 5011 can be further protruded from an external wall of the mouth piece 60. The restricting recessed groove 601 is recessed from the sidewall of the through hole 501 accordingly, the loss of the mouth piece 60 can also be prevented.

Referring to FIG. 2 again, in the illustrated embodiment, the liquid injection protective mechanism further includes a movable ring 403 rotatably sleeved on an outer of the connecting assembly 40, the connecting assembly 40 is hidden within the movable ring 403. Specifically, the movable ring 403 is rotatably sleeved on an outer of the connecting base 411, it is difficult for user to take down the connecting assembly 40 by handling the movable ring 403 to allow the connecting assembly 40 to rotate relative to the housing 10, thereby a disassembly of the connecting assembly 40 can be avoided, such that children cannot casually disassemble the connecting assembly 40 and contact the e-cigarette liquid in the liquid storage chamber 101. In a specific embodiment, the movable ring 403 shields an external circumferential surface of the connecting base 411 completely, causing user difficult to apply a force to the connecting base 411. Only if the cover 50 is reversed relative the connecting assembly 40, a force can be easily applied to the cover 50 to take down the connecting assembly 40, thereafter, operation such as a replacement of the atomizing head 20 can be realized.

It can be understood that, when user performs a liquid injection, the first locking assembly between the cover 50 and the connecting assembly 40 is firstly unlocked, causing the locking member to separate from the locking hole, and then the second locking assembly between the cover 50 and the connecting assembly 40 is unlocked, causing the first connecting portion 422 to separate from the second connecting portion 421, thereby the cover 50 can be reversed to reveal the liquid intake hole 402 of the connecting assembly 40, a liquid injection operation can thereby be conducted. After the liquid injection operation is accomplished, the locking procedure is the inverse of the unlocking procedure, which is not specific described herein.

It can be understood that, in alternative embodiment not shown, only one of the first locking assembly and the second locking assembly should be configured.

In the atomizer 100 provided by the first embodiment of the present disclosure, a locking relationship between the connecting assembly 40 and the cover 50 should be removed first, and then the cover 50 can be pushed to reveal the liquid injection hole 402 by an operation combing a translational pushing and a reversing action, thus the liquid injection operation can be performed. Therefore, the open procedure of the liquid injection hole 402 is complicated, the liquid injection protective mechanism is childproof.

In the electronic cigarette provided by the first embodiment of the present disclosure, all technical features of aforementioned atomizer 100 are included, thus the electronic cigarette has a technical advantage same as that in the aforementioned atomizer 100.

### The Second Embodiment

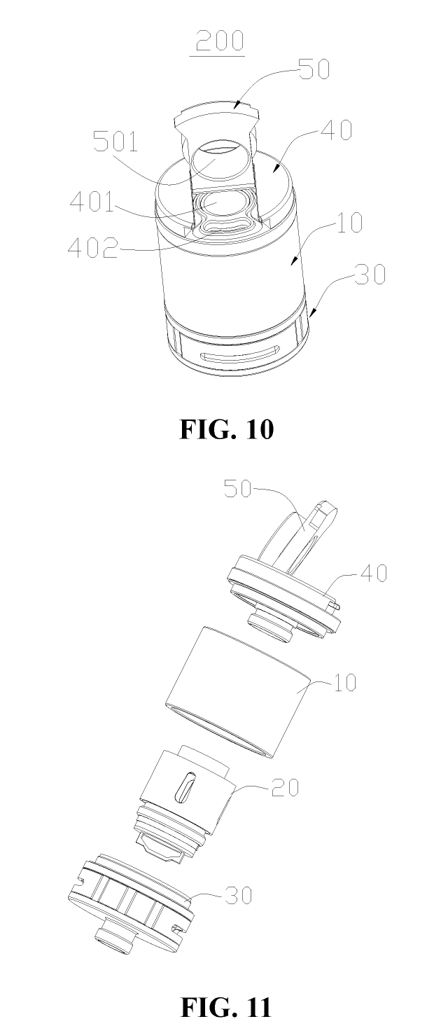

Referring to FIG. 10 and FIG. 11, an electronic cigarette is provided by the second embodiment of the present disclosure. The electronic cigarette includes an atomizer 200 and a power source mechanism (not shown) electrically connected to the atomizer 200. Compared to the electronic cigarette of the first embodiment, the difference is that, the structure and the matching mode of the connecting assembly 40 and the cover 50 of the atomizer 200 are different from that in the first embodiment, the housing 10, the atomizing head 20 and the base seat assembly 30 are same as that in the first embodiment, which is not specific described herein.

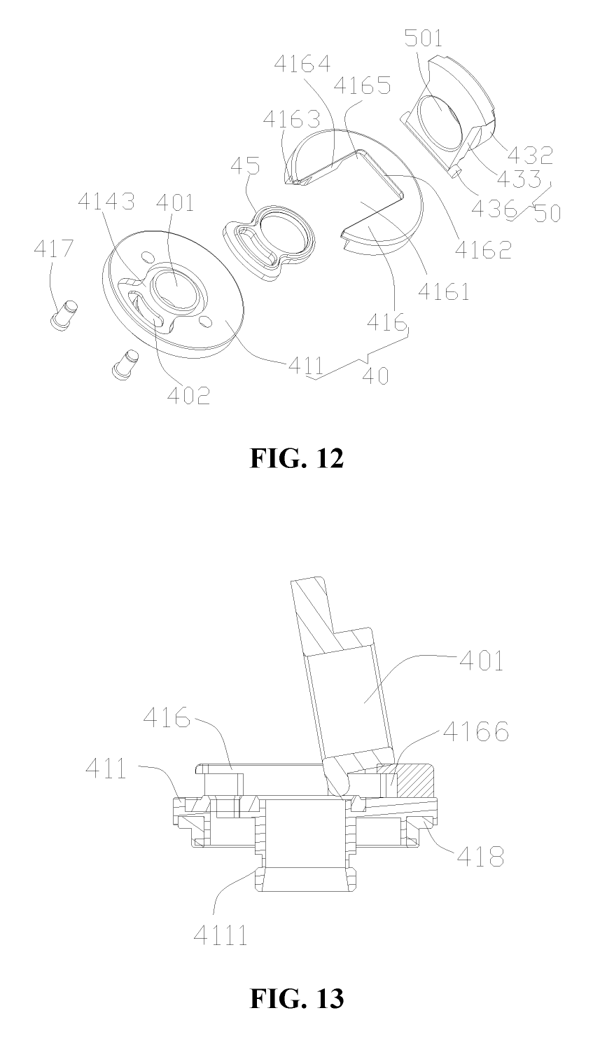

Referring to FIG. 12 and FIG. 13, in the illustrated embodiment, the connecting assembly 40 includes a connecting base 411 and the pressing cover 416 covering above the connecting base 411. The aerosol exhaust hole 401 and the liquid injection hole 402 are defined on the connecting base 411 successively and extend outwardly from a center of the connecting base 411 along a radial direction. The connecting base 411 is positioned on an end of the housing 10 away from the base seat assembly 30. The connecting base 411 is provided with a connecting post 4111. The connecting post 4111 is hollow and protrudes downwardly from a surface of the connecting base 411 away from the pressing cover 416. The connecting post 4111 is plugged into an end of the atomizing sleeve 21 away from the base seat assembly 30, for fixing the connecting assembly 40 to the atomizing head 20, and allowing the aerosol flow in the atomizing chamber 201. Further, the connecting assembly 40 further includes a sealing ring 418 positioned between the connecting base 411 and the atomizing head 20, for preventing a leakage of the e-cigarette liquid.

The pressing cover 416 defines an U-shaped cutout 4161 with an opening at an end, the U-shaped cutout 4161 is recessed from an edge of a side of the pressing cover 416, the aerosol exhaust hole 401 and the liquid injection hole 402 are revealed and received in the cutout 4161. Specifically, the cutout 4161 includes a connecting wall 4162 positioned on a side of the aerosol exhaust hole 401 away from the liquid injection hole 403, and two sidewalls 4164 positioned on opposite ends of the connecting wall 4162, the two sidewalls 4146 extend toward a same direction. A surface of the connecting wall 4162 adjacent to the connecting base 411 defines a recessed groove 4166, the recessed groove 4166 is recessed from the connecting wall 4162 along a direction away from the aerosol exhaust hole 401, thereby a receiving separation having a determined height is formed between the pressing cover 416 and the connecting base 411. At the same time, each sidewall 4164 defines a translational groove 4165 at an end of the sidewall 4164 connecting with the connecting wall 4162, the translation groove 4165 is recessed from a surface of the sidewall 4164 adjacent to the connecting base 411, the two surfaces of the two sidewalls 4164 defining the translational grooves 4165 are opposite to each other. The translational groove 4165 is fluidly communicating with the cutout 4161 and the recessed groove 4166. At the time, the two translational grooves 4165 and the two grooves 4166 cooperatively constitute a U-shaped groove surrounding the cutout 4161 and having an opening direction same as that of the cutout 4161.

When an end of the cover 50 is received and positioned in the recessed groove 4166, the cover 50 is positioned on the locking position, the cover 50 cannot be reversed relative to the connecting assembly 40. When the cover 50 is moved along the translational groove 4165 to enable the end of the cover 50 extend out of the recessed groove 4166, the cover 50 is positioned on the unlocking position, the cover 50 can be reversed.

Specifically, the cover 50 includes a movable member 433 and a rotation shaft 436 with opposite ends extending out of opposite ends of a side of the movable member 433. The movable member 433 has a substantially elongated plate shape, it has a shape matching the shape of the cutout 4161. The through hole 501 extends through the movable member 433, and the sidewall of the through hole 501 protrudes out of the surface of the movable member 433 away from the connecting base 411. When the cover 50 is positioned on the locking position, the rotation shaft 436 is latched within the recessed groove 4166, and the sidewall of the through hole 501 resists the sidewall of the recessed groove 4166, thereby preventing the cover 50 to reverse around the rotation shaft 436. When the cover 50 is exerted by an external force, and moves toward the unlocking position from the locking position, the rotation shaft 436 moves into the translational groove 4165 from the recessed groove 4166, causing the sidewall of the through hole 501 and the sidewall of the cutout 4166 are bent away from each other to form a rotation space for the movable member 433 being reversible around the rotation shaft 436 relative to pressing cover 416. Thus, by a reversing of the cover 50, the cover 50 is opened relative to the connecting assembly 40, revealing the liquid injection hole 402, facilitating for user to conduct a liquid injection action. The cover 50 can cover the connecting assembly 40 and is pushed to return to the locking position from the unlocking position, after a liquid injection is accomplished. When the movable member 433 is positioned on the locking position, it cannot directly be reversed. When a translational pushing is preformed to remove an interference function between the movable member 433 and the cover 416, and then the movable member 433 can be reversed, an opening of the cover 50 in the using procedure of the electronic cigarette is avoided, and it is childproof.

The cover 50 further includes a covering portion 432 positioned on an end of the movable member 433 opposite to the rotation shaft 436. The covering portion 432 extends to opposite sides of the movable member 433. A width of the covering portion 432 is greater than that of the cutout 4161. In the illustrated embodiment, the covering portion 432 has a sector shape spreading to opposite sides of the movable member 433. At the same time, the pressing cover 416 defines a locking groove 4163 on a bottom of an end of the pressing cover 416 away from the connecting wall 4162, the locking groove 4163 is fluidly communicating with the cutout 4161. The locking groove 4163 has a width greater than that of the cutout 4161, i.e. the locking groove 4163 and the cutout 4161 cooperatively constitute a stepped groove with a wider top and a narrow bottom. Therefore, when the cover 50 is pushed to the locking position relative to the connecting assembly 40, the covering portion 432 is received in the locking groove 4163 and resists the bottom wall of the locking groove 4163, for preventing the cover 50 to be pushed continuously. On the other hand, the upper surface of the covering portion 432 resists the upper wall of the locking groove 4163, thereby a further reversal of the cover 50 relative to the connecting assembly 40 can be avoided. When the cover 50 is pushed to the unlocking position relative to the connecting assembly 40, the covering portion 432 is moved outside the locking groove 4163 via the opening of the locking groove 4163, enabling the cover 50 to be reversed relative to the connecting assembly 40.

It can be understood that, the locking groove 4163 forms a first connecting portion, the covering portion 432 forms a second connecting portion, the first connecting portion and the second connecting portion cooperatively constitutes a second locking assembly. When the cover 50 is moved to enable the covering portion 432 to be received in the locking groove 4163, the cover 50 is locked. When the cover 50 is moved to enable the covering portion 432 to separate from the locking groove 4163, the cover 50 is unlocked.

According to a specific embodiment, the liquid injection hole 402 is an arc-shaped opening surrounding a part of the outer the aerosol exhaust hole 401, the liquid injection hole 402 not only has a shape matching the sector shape of the covering portion 432, but also has an greater size, facilitating for a liquid injection operation.

In the illustrated embodiment, in order to guarantee an air-tightness between the pressing cover 416 and the cover 50, a sealing groove 4143 is defined on the connecting base 411 and positioned a periphery of the aerosol exhaust hole 401 and the liquid injection hole 402. The connecting assembly 40 further includes a sealing pad 45, the sealing pad 45 is received in the sealing groove 4143 and heretically seals the periphery of the aerosol exhaust hole 401 and the liquid injection hole 402.

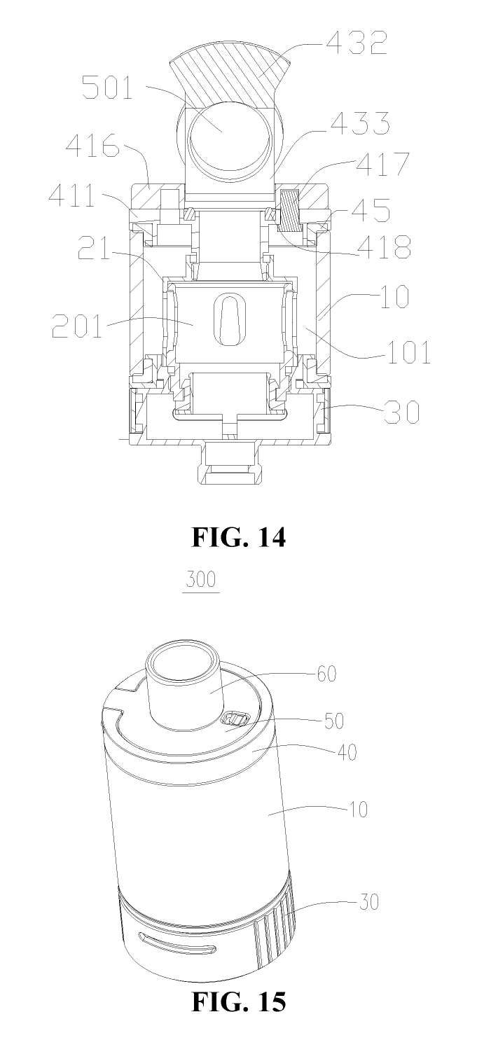

Further referring to FIG. 14, in the illustrated embodiment, the connecting assembly 40 further includes a pin 417, the pin 417 extends beneath the connecting base 411 and is fixed to the pressing cover 416, for achieving a fixation relationship between the connecting base 411 and the pressing cover 416. It can be understood that, in alternative embodiment not shown, the fixation mode between the pressing cover 416 and the connecting base 411 can be determined according to a requirement, or the two elements are integrally formed, which is not limited hereby.

Further, in order to prevent opening the liquid injection hole 402 causally and contact the e-cigarette liquid in the liquid storage chamber 101 when children takes the atomizer 200, in addition to the second locking assembly, the atomizer 200 can further include a first locking assembly, the structures and engagements of the first locking assembly are same as that in the first embodiment, which is not specific described hereby.

It can be understood that, the connecting assembly 40, the cover 50, the first locking assembly, and the second locking assembly cooperatively constitute a liquid injection protective mechanism.

When an end of the locking member extends through the locking hole and is connected to the connecting assembly 40. The movable stroke of the cover 50 is restricted by the locking member, thereby preventing moving the cover 50 to reveal the liquid injection hole 402.

It can be understood that, when user perform a liquid injecting, the first locking assembly between the cover 50 and the connecting assembly 40 is required to be removed first, enabling the locking member to separate from the locking hole, and then the second locking assembly between the cover 50 and the connecting assembly 40 is removed, enabling the covering portion 432 to separate from the locking groove 4163, and then the liquid injection can be conducted.

In the atomizer 200 provided by the second embodiment of the present disclosure, the locking relationship between the connecting assembly 40 and the cover 50 applied by the locking member is required to be removed first, and then the cover 50 reveals the liquid injection hole 402 by an operation combing a translational movement and a reversing movement. Therefore, the opening procedure of the liquid injection hole 402 is relative complicated, it therefore is childproof. At the same time, compared to the first embodiment, the atomizer 200 of the second embodiment 200 has a relative simple structure, which facilitates to manufacture.

In the electronic cigarette provided by the second embodiment of the present disclosure, because all the technical features in aforementioned atomizer 200 are included, thus the electronic cigarette has technical advantages same as that of the atomizer 200.

### The Third Embodiment

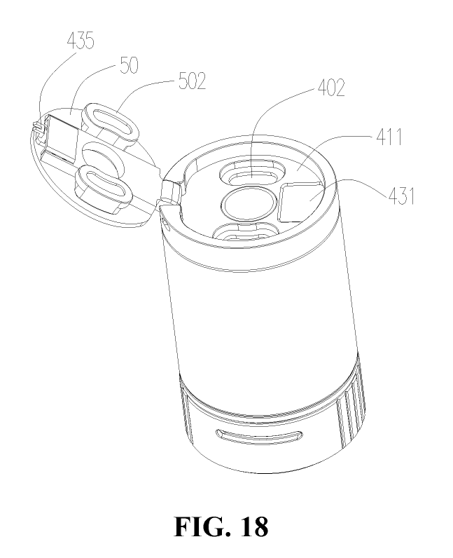

Referring to FIG. 15 and FIG. 18, an electronic cigarette is provided by the third embodiment of the present disclosure, the electronic cigarette includes an atomizer 300 and a power source mechanism (not shown) electrically connected to the atomizer 300. Wherein, the atomizer 300 includes a housing 10, an atomizing head 20 received in the housing 10, a base seat assembly 30 connected to an end of the housing 10, a connecting assembly 40 connected to an end of the housing 10 opposite to the base seat assembly 30, and a cover 50 covering the connecting assembly 40, the power source mechanism is detachably mounted on the base seat assembly 30 and is electrically connected to the atomizing head 20. The housing 10 stores the e-cigarette liquid therein, and can supply the e-cigarette liquid to the atomizing head 20. The atomizing head 20 is driven by the power source mechanism to heat the e-cigarette liquid to generate aerosol for user to inhale.

Wherein, the structures of the housing 10, the atomizing head 20, and the base seat assembly 30 are same as that in the first embodiment, which is not specific described herein.

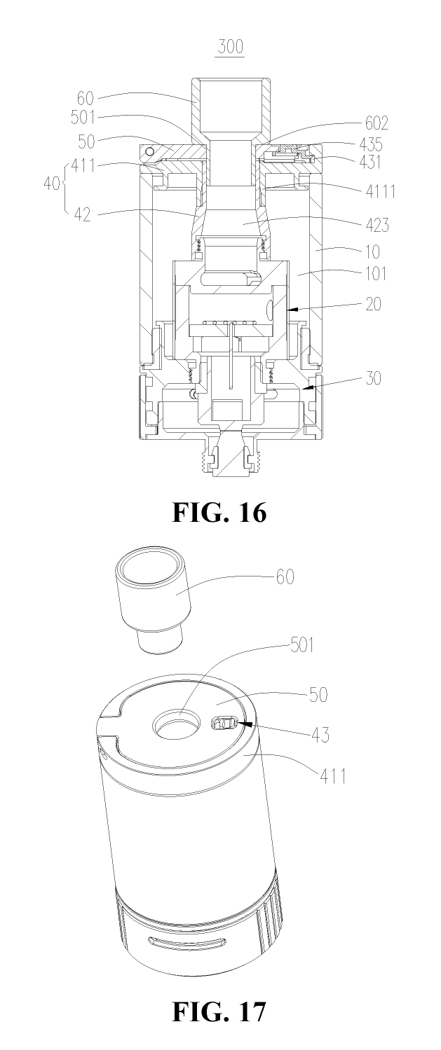

Referring to FIG. 16, in the illustrated embodiment, the connecting assembly 40 includes a connecting base 411 connected to an upper end of the housing 10, and a ventilation member 42 positioned within the housing 10 and connected to the lower end of the connecting base 411.

The connecting base 411 is plugged in the upper end of the housing 10, the connecting base 411 has a substantially hollow cylindrical structure with an opening at an upper end. The connecting base 411 is provided with a connecting post 4111 at the center of a bottom of the connecting base 411 for the aerosol to flow out. The upper end of the connecting post 4111 extends through the bottom of the connecting base 411, the lower end of the connecting post 4111 extends downwardly into the housing 10 along an axial direction of the atomizer 300. The bottom of the connecting base 411 defines two liquid injection holes 402 on opposite sides of the connecting post 4111, both the two liquid injection holes 402 are fluidly communicating with the liquid storage chamber 101. User can inject e-cigarette liquid into the liquid storage chamber 101 via the liquid injection holes 402. It can be understood that, in alternative embodiment not shown, a number of the liquid injection hole 402 can be one or more than two.

The ventilation member 42 has a substantially hollow cylindrical structure with two openings at opposite ends. The upper end of the ventilation member 42 is detachably connected to the connecting base 411. The lower end of the ventilation member 42 is detachably connected to the atomizing head 20. Specifically, the upper end of the ventilation member 42 is tightly sleeved in the inner of the connecting post 4111, the lower end of the ventilation member 42 is connected to the atomizing head 20 via threads. The interior chamber of the ventilation member 42 forms an aerosol exhaust passage 423, when in use, the aerosol generated in the atomizing head 20 flows into user's mouth via the aerosol exhaust passage 423.

It can be understood that, in alternative embodiment not shown, the upper end of the ventilation member 42 can be connected to the connecting post 4111 via a threaded connection or latching, the lower end of the ventilation member 42 can be connected to the atomizing head 20 via a threaded connection or latching, which is not limited hereby.

It can be understood that, in alternative embodiment not shown, the upper end of the ventilation member 42 is tightly sleeved on an outer of the connecting post 4111, thus a connection between the ventilation member 42 and the connecting post 4111 can also be achieved.

It can be understood that, in alternative embodiment not shown, the ventilation member 42 can be omitted, at the time, the atomizing head 20 is directly and detachably connected to the connecting post 4111.

Referring to FIG. 16 again, the cover 50 has a substantially disc structure, the edge of the cover is hinged to the top of the connecting base 411, enabling the cover 50 to be reversed relative to the connecting base 411, thereby causing the cover 50 to cover or open the opening of the upper end of the connecting base 411, such that the liquid injection hole 402 can be covered or opened.

In order to prevent opening the liquid injection hole 402 causally and contacting the e-cigarette liquid when children takes the atomizer 300, the atomizer 300 further includes a first locking assembly, the first locking assembly includes a locking hole defines on the cover 50 and a locking member detachably connected to the connecting assembly 40. When an end of the locking member extends through the locking hole and is connected to the connecting assembly 40, the movable stroke of the cover 50 is restricted by the locking member, thereby preventing moving the cover 50 to reveal the liquid injection hole 402.

In the illustrated embodiment, the locking hole is a through hole 501 defined on a center the cover 50, the locking member is a mouth piece 60 extending through the through hole 501 and connected to the connecting assembly 40. Specifically, the mouth piece 60 has a substantially hollow cylindrical structure with two openings at opposite ends, after the lower end of the mouth piece 60 extends through the through hole 501 of the cover 50, the lower end of the mouth piece 60 is tightly sleeved within the interior of the ventilation member 42, thereby a connection between the mouth piece 60 and the connecting assembly 40 is established. It can be understood that, in alternative embodiment not shown, when the upper end of the ventilation member 42 is tightly sleeved on an outer of the connecting post 4111, or when the ventilation member 42 is omitted, the lower end of the mouth piece 60 is tightly sleeved within the interior of the connecting post 4111, thereby a connection between the mouth piece 60 and the connecting assembly 40 is established. It can also be understood that, in alternative embodiment not shown, the mouth piece 60 can be connected to the connecting assembly 40 by a threaded connection or latching.

In the illustrated embodiment, the mouth piece 60 has an outer diameter equal to an inner diameter of the though hole 501 of the cover 50. Specifically, the outer diameter of the portion of the mouth piece 60 matching the through hole 501 is equal to the inner diameter of the through hole 501. When the mouth piece 60 is connected to the connecting assembly 40, because the outer diameter of the portion of the mouth piece 60 matching the through hole 501 is equal to the inner diameter of the through hole 501, thus a movable space on the position where the mouth piece 60 matching the through hole 501 is eliminated, the cover 50 cannot be reversed relative to the mouth piece 60. Further, due to a connecting relationship between the mouth piece 60 and the connecting assembly 40, the cover 50 and the mouth piece 60 cannot be reversed relative to the connecting base assembly, the cover 50 is locked.

Further, the external wall of the mouth piece 60 is provided with a resisting edge 602, when the mouth piece 60 is connected to the connecting assembly 40, the resisting edge 602 can resist the cover 50, thereby the cover 50 is restricted between the connecting base 411 and the resisting edge 602, preventing the cover 50 to be reversed, the cover 50 is locked. Further, the resisting edge 602 is further configured to provide a restricting function for the assembly of the mouth piece 60. When the resisting edge 602 resists the cover 50, it means that the mouth piece 60 is mounted in place. In the illustrated embodiment, the resisting edge 602 is formed by a shrinkage of a neck of the mouth piece 60. It can be understood that, in alternative embodiment not shown, the resisting edge 602 can further be formed by extending outwardly along a radial direction of the mouth piece 60 from the circumferential surface of the mouth piece 60.

It can be understood that, in the two selections: the outer diameter of the portion of the mouth piece 60 matching the through hole 501 is equal to the inner diameter of the through hole 501, or, the mouth piece 60 is provided with a resisting edge 602 resisting the cover 50, at least one selection is required to be met, i.e. when the mouth piece 60 extends through the through hole 501 and is connected to the connecting assembly 40, the movable stroke of the cover 50 is restricted by the mouth piece 60, thereby the cover 50 is prevented to move to reveal the liquid injection hole 402. Only if the mouth piece 60 is dismantled, the cover 50 can be further moved to reveal the liquid injection hole 402. It can be understood that, the two manners: the outer diameter of the portion of the mouth piece 60 matching the through hole 501 is equal to the inner diameter of the through hole 501, and, the mouth piece 60 is provided with a resisting edge 602 resisting the cover 50, can also be applied to the electronic cigarette of the first embodiment and the second embodiment, thereby only if the mouth piece 60 is dismantled, the cover 50 can be further moved to reveal the liquid injection hole 402.

It should be noted that, the mouth piece is a conventional element for atomizer, user should hold the mouth piece in mouth to smoke. In the illustrated embodiment, by an improvement of the mouth piece 60, the mouth piece 60 not only has a smoking function, but also locks the cover 50, preventing the liquid injection hole 402 to be opened causally, such that merely a minor change to the structure without increases of other parts is conducted, childproof function is thus realized, it is cost saving.

Referring to FIG. 16 and FIG. 18, in the illustrated embodiment, in order to improve a reliability of the childproof function, for preventing the cover 50 to be reversed after the locking member is dismantled by children, a second locking assembly is positioned between the connecting base 411 and the cover 50. Specifically, the second locking assembly includes a first connecting portion and a second connecting portion, the first connecting portion is a first fixing member 431 positioned on the connecting base 411, the second connecting portion is a second fixing member 435 positioned on the cover 50. In the illustrated embodiment, the first fixing member 431 is a latching groove positioned on the bottom of the internal circumferential wall of the connecting base 411, the second fixing member 435 is a latching member is slidably positioned on the edge of the cover 50 and corresponding to the latching groove. The latching member is partially exposed out of the upper end surface of the cover 50, for user to conduct a sliding action of the latching member, causing an end of the latching member to latch with or separate from the latching groove. When the cover 50 is reversed and covers the connecting base 411, by a latching between the latching member and the latching groove, the covering applied to the connecting base 411 by the cover 50 is maintained, the cover 50 is prevented from being reversed causally. Therefore, even if the locking member is dismantled, the cover 50 cannot be reversed causally, the second locking assembly should be further unlocked if a liquid injection is required.

It can be understood that, in alternative embodiment not shown, the first fixing member 431 can also be a first magnetic member positioned on the bottom of the connecting base 411, the second fixing member 435 is a second magnetic member positioned on the lower surface of the cover 50 and corresponding to the first magnetic member. When the cover 50 covers the connecting base 411, the first magnetic member and the second member are mutually absorbed, thereby the covering applied to the connecting base 411 by the cover 50 is maintained.

Referring to FIG. 18, in order to improve an air-tightness of the liquid injection hole 402 applied by the cover 50, when the cover 50 covers the connecting base 411, the cover 50 is provided with a sealing member 502 protruding from the lower surface of the cover 50 and corresponding to the liquid injection hole 402. When the cover 50 covers the connecting base 411, the sealing member 502 is capable of latching into the liquid injection hole 402 for sealing the liquid injection hole 402, thereby an e-cigarette liquid leakage is avoided. It can be understood that, the sealing member 502 is made of sealing materials such as the silica gel or rubber, which is not limited hereby. It can be understood that, when the sealing member 502 latches into the liquid injection hole 402, by a latching between the sealing member 502 and the liquid injection hole 402, a covering of the cover 50 applied to the connecting base 411 is maintained. At the time, as the parts of the second locking assembly, the first fixing member 431 and the second fixing member 435 can be omitted.

It can be understood that, the cover 50, the connecting assembly 40, the first locking assembly, and the second locking assembly cooperative constitute a liquid injection protective mechanism. When an end of the locking member extends through the locking hole and is connected to the connecting assembly 40, the movable stroke of the cover 50 is restricted by the locking member, for preventing moving the cover 50 to reveal the liquid injection hole 402.

In the atomizer 300 provided by the third embodiment of the present disclosure, when user want to perform a liquid injection, referring to FIG. 17, the mouth piece 60 is required to be dismantled first, and then the latching relationship between the first fixing member 431 and the second fixing member 435 is removed, at the time, the locking relationship between the cover 50 and the connecting base 411 is removed, referring to FIG. 18, the cover 50 is reversed, to enable the liquid injection hole 402 to be opened. Because the liquid injection hole 402 can only be opened by a complicated operation procedure, thus children cannot open the liquid injection hole 402 causally, and a contact and swallow of the e-cigarette liquid for children can be avoided, it is childproof. It can be understood that, referring to a configuration in the first embodiment, a restricting recessed groove 601 is defined on the mouth piece 60 or the cover 50, the other one is provided with a restricting protrusion 5011, by an engagement between the restricting recessed groove 601 and the restricting protrusion 5011, the movable stroke of the mouth piece 601 is restricted, therefore, the mouth piece 60 cannot lost. When a liquid injection is required, merely the mouth piece 60 is drawn upward to enable the lower end of the mouth piece 60 to be received in the through hole 501.

In the electronic cigarette provided by the third embodiment of the present disclosure, all technical features of aforementioned atomizer 300 are included, thus the electronic cigarette has a technical advantage same as that in aforementioned atomizer 300.

### The Fourth Embodiment

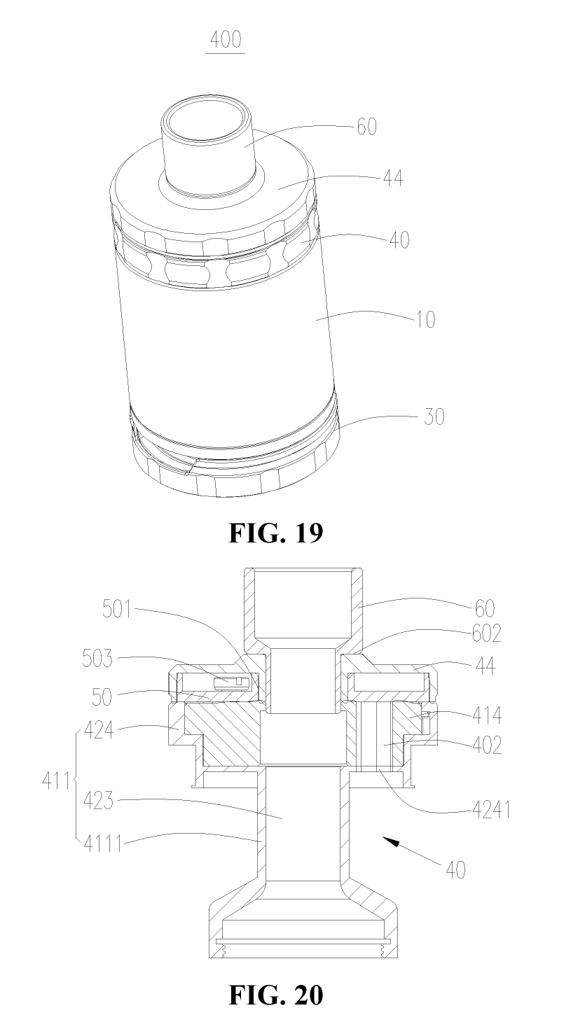

Referring to FIG. 19 through FIG. 22, an electronic cigarette is provided by the fourth embodiment of the present disclosure. The electronic cigarette includes an atomizer 400 and a power source mechanism (not shown) electrically connected to the atomizer 400. Compared to the electronic cigarette of the third embodiment, the difference is that, the structure and the matching mode of the connecting assembly 40 and the cover 50 of the atomizer 400 are different from that in the third embodiment, the housing 10, the atomizing head 20 and the base seat assembly 30 are same as that in the third embodiment, which is not specific described herein.

Specifically, in the illustrated embodiment, the connecting assembly 40 includes a connecting base 411 connected to the upper end of the housing 10 and a lining member 414 received in the connecting base 411.

Referring to FIG. 20, in the illustrated embodiment, the connecting base 411 includes a connecting tube 424 connected to an upper end of the housing 10 and a connecting post 4111 extending through the bottom of the connecting tube 424. The connecting tube 424 defines a receiving chamber with an opening at an upper end, the connecting tube 424 defines a perforation 4241 at a bottom, the perforation 4241 is positioned on a side of the connecting post 4111. The lower end of the connecting post 4111 extends into the housing 10 and is connected to the atomizing head 20, the interior chamber of the connecting post 4111 forms an aerosol exhaust passage 423, the aerosol generated in the atomizing head 20 flows into user's mouth via the aerosol exhaust passage 423.

The lining member 414 has a substantially cylindrical structure with opposite ends cutting through each other. The lining member 414 is received in the connecting tube 424, the chambers of the opposite ends of the lining member are fluidly communicating with the aerosol exhaust passage 423. The lining member 414 defines a liquid injection hole 402 corresponding to the perforation 4241. The liquid injection hole 402 is fluidly communicating with the liquid storage chamber 101 via the perforation 4241, user can inject e-cigarette liquid into the liquid storage chamber 101 via the liquid injection hole 402.

Referring to FIG. 20, in the illustrated embodiment, the cover 50 includes a covering plate having a substantially disc structure. The covering plate is provided with a rotation shaft 503, the covering plate is rotatably mounted on the lining member 414 via the rotation shaft 503. A rotation center of the covering plate is staggered from a central axial axis of the lining member 414. During the rotation procedure, the lower end surface of the covering plate contacts the upper end surface of the lining member 414, and the covering plate can open or close the liquid injection hole 402.

In the illustrated embodiment, the cover 50 further includes a rotating member 44 tightly sleeved on an outer of the covering plate, when the rotating member 44 is rotated, the rotating member 44 can drive the covering plate to rotate. The rotating member 44 provides a shielding function for the covering plate, enabling the atomizer 400 to have an attractive appearance. It can be understood that, in alternative embodiment, the rotating member 44 can further be omitted, user merely rotates the covering plate.

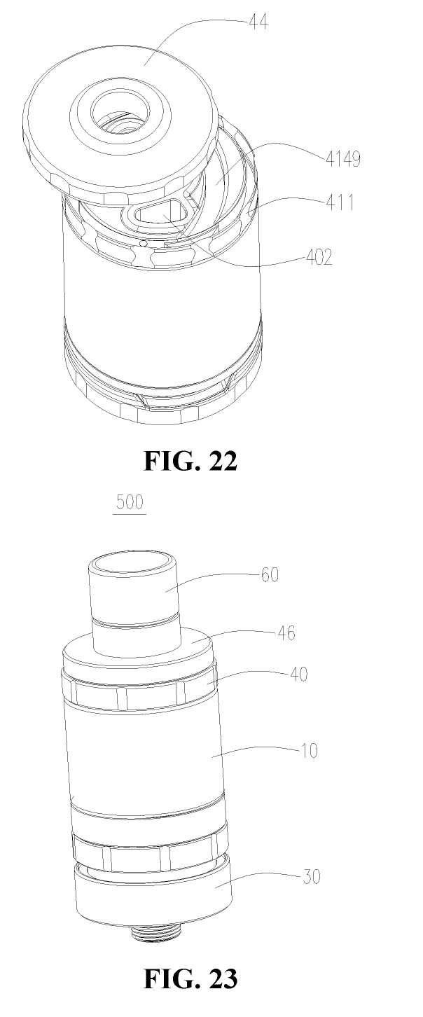

Referring to FIG. 22, when conducting a liquid injection, the rotating member 44 is rotated, the rotating member 44 drives the covering plate to rotate around the rotation shaft 503, the covering plate is staggered from the lining member 414 gradually, until the liquid injection hole 402 is revealed. At the time, the liquid injection hole 402 is opened, user can conduct a liquid injection operation.

In order to provide a stable rotation of the cover 50 when the cover 50 rotates, the lining member 414 defines an arc-shaped guiding groove 4149 surrounding a center of the rotation shaft 503. The lower surface of the covering plate is provided with a guiding protrusion (not shown) corresponding to the guiding groove 4149. During the rotation procedure of the cover 50, the guiding protrusion can slide along the guiding groove 4149.

In order to improve the air-tightness of the liquid injection hole 402 applied by the cover 50, the covering plate is made of elastic materials such as the silica gel or rubber.

Further, the atomizer 400 further includes a first locking assembly. It can be understood that, the connecting assembly 40, the cover 50, and the first locking assembly cooperatively constitute a liquid injection protective mechanism. The first locking assembly further includes a locking hole positioned on the cover 50, and a locking member extending through the locking hole and detachably connected to the connecting assembly 40. In the illustrated embodiment, the locking hole is a through hole 501 defined on the cover 50, the locking member is a mouth piece 60 extending through the through hole 501 and connected to the connecting assembly 40. It can be understood that, in the illustrated embodiment, because the covering plate is wrapped up by the rotating member 44, therefore, the through hole 501 is defined on the rotating member 44.

The mouth piece 60 has a substantially hollow cylindrical structure with two openings at opposite ends. The mouth piece 60 extends through the through hole 501 of the cover 50 and is tightly plugged in the lining member 414. At the same time, the liquid injection hole 402 is sealed by the cover 50, and the mouth piece 60 is fluidly communicating with the aerosol exhaust passage 423, user can aerosol by hold the mouth piece 60 in mouth.

When the mouth piece 60 is connected to the connecting assembly 40, the rotation stroke of the cover 50 is restricted by the mouth piece 60, the mouth piece 60 has an outer diameter equal to an inner diameter of the though hole 501 of the cover 50. Specifically, the outer diameter of the portion of the mouth piece 60 matching the through hole 501 is equal to the inner diameter of the through hole 501, because the movable space on the position where the mouth piece 60 matching the through hole 501 is eliminated, therefore, the cover 50 cannot be reversed relative to the mouth piece 60, and the cover 50 is locked. When the mouth piece 60 is dismantled, the cover 50 is then rotatable.

Further, the external wall of the mouth piece 60 is provided with a resisting edge 602, when the mouth piece 60 is connected to the connecting assembly 40, the resisting edge 602 can resist the cover 50, thereby providing a restricting function for the assembly of the mouth piece 60, i.e. when the resisting edge 602 resists the cover 50, it means that the mouth piece 60 is mounted in place. In the illustrated embodiment, the resisting edge 602 is formed by a shrinkage of the neck of the mouth piece 60. It can be understood that, in alternative embodiment not shown, the resisting edge 602 can further be formed by extending outwardly from the circumferential surface of the mouth piece 60 along a radial direction of the mouth piece 60.

The liquid injection protective mechanism further includes a second locking assembly, the second locking assembly includes a first connecting portion and a second connecting portion. It can be understood that the configuration can be referred to that in the third embodiment. The first connecting portion is a latching groove positioned on the connecting base 411. The second connecting portion is a latching member slidably positioned on the cover 50 and corresponding to the latching groove. After the locking member of the first locking assembly is detached from the locking hole, it is required that the latching member is separated from the latching groove, and thus the liquid injection hole 402 can be opened by a rotation of the cover 50.

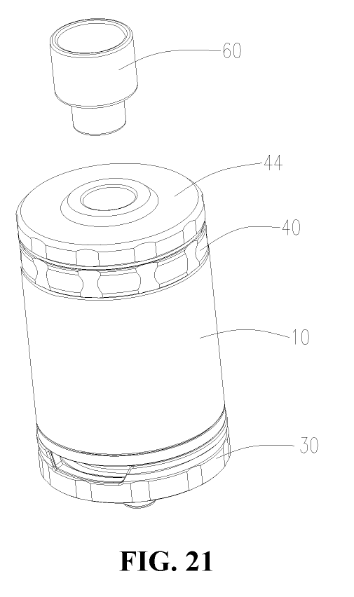

In the atomizer 400 provided by the fourth embodiment of the present disclosure, when user want to perform a liquid injection action, referring to FIG. 21, the mouth piece 60 is required to be dismantled first, and then the latching relationship between the cover 50 and the connecting assembly 40 is removed, referring to FIG. 22, the rotating member 44 is rotated, to enable the cover to be driven by the rotating member 44 to rotate, thereby opening the liquid injection hole 402. Because the liquid injection hole 402 can only be opened by a complicated operation procedure, thus children cannot easily open the liquid injection hole 402, and a contact and swallow of the e-cigarette liquid for children can be avoided, it is therefore childproof. It can be understood that, referring to a configuration in the first embodiment, a restricting recessed groove 601 is defined on the mouth piece 60 or the cover 50, the other one is provided with a restricting protrusion 5011, by an engagement between the restricting recessed groove 601 and the restricting protrusion 5011, the movable stroke of the mouth piece 601 can be restricted, therefore, the mouth piece 60 cannot lose. When a liquid injection is required, merely the mouth piece 60 is drawn upward to enable the lower end of the mouth piece 60 to be received in the through hole 501.

In the electronic cigarette provided by the fourth embodiment of the present disclosure, all technical features of aforementioned atomizer 400 are included, thus the electronic cigarette has a technical advantage same as that in aforementioned atomizer 400.

### The Fifth Embodiment

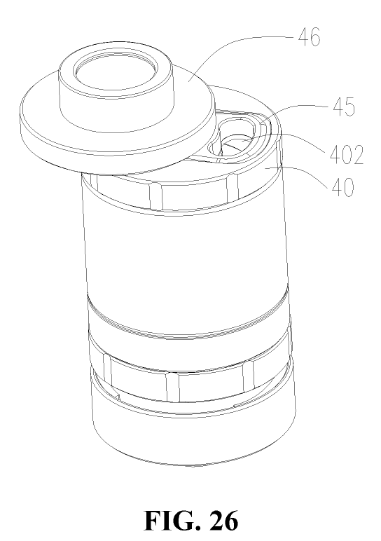

Referring to FIG. 23 and FIG. 26, an electronic cigarette is provided by the fifth embodiment of the present disclosure. The electronic cigarette includes an atomizer 500 and a power source mechanism (not shown) electrically connected to the atomizer 500. Compared to the electronic cigarette of the third embodiment, the difference is that, the structure and the matching mode of the connecting assembly 40 and the cover 50 of the atomizer 500 are different from that in the third embodiment, the housing 10, the atomizing head 20 and the base seat assembly 30 are same as that in the first embodiment, which is not specific described herein

Specifically, in the illustrated embodiment, the connecting assembly 40 includes a connecting base 411 connected to the upper end of the housing 10. The cover 50 is positioned on the upper end of the connecting base 411 and is slidable relative to the connecting base 411. The mouth piece 60 extends through the cover 50 and is connected to the connecting base 411.

The connecting base 411 is plugged in the upper end of the housing 10, the connecting base 411 has a substantially hollow cylindrical structure with an opening at a lower end. The connecting base 411 is provided with a connecting post 4111 at the center of a bottom of the connecting base 411 for the aerosol to flow out. The upper end of the connecting post 4111 defines one liquid injection hole 402 at a side of the connecting post 4111. The liquid injection hole 402 is fluidly communicating with the liquid storage chamber 101, user can inject e-cigarette liquid into the liquid storage chamber 101 via the liquid injection hole 402.

In the illustrated embodiment, the cover 50 includes a covering plate, the covering plate has a substantially hollow cylindrical structure with two openings at opposite ends, the chambers having opening on opposite ends of the covering plate are fluidly communicating with the connecting post 4111. During the sliding procedure of the covering plate, the lower end surface of the covering plate contacts the upper end surface of the connecting base 411, and the liquid injection hole 402 can be opened or closed.

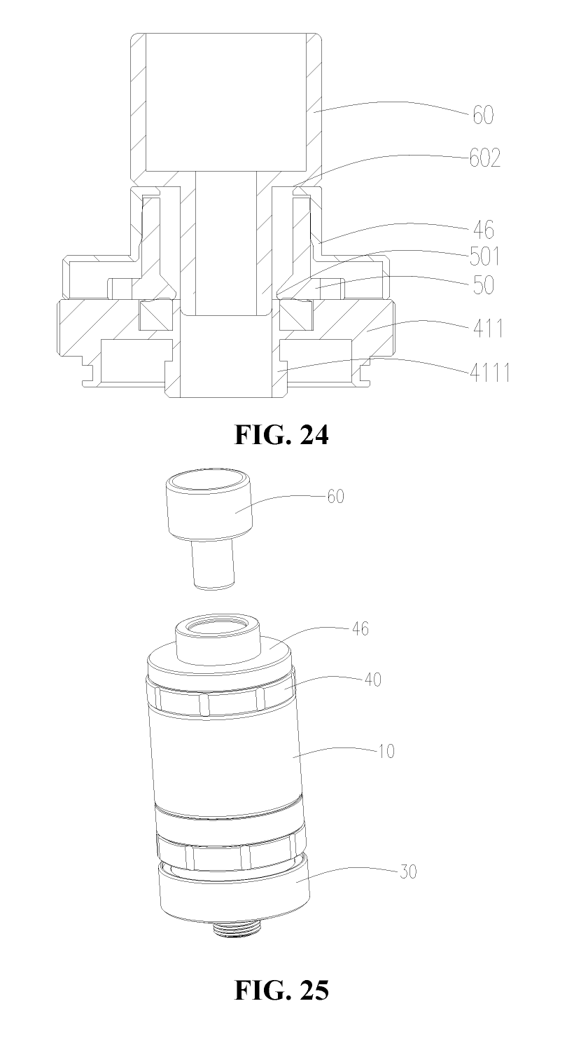

Referring to FIG. 24, in the illustrated embodiment, the cover 50 further includes a sliding member 46 tightly sleeved on an outer of the covering plate, specifically, the sliding member 46 has a substantially cylindrical structure with a cross-section having a convex shape. When the sliding member 46 slides, the sliding member 46 can slide together with the covering plate. The sliding member 46 provides a shielding function for the covering plate, enabling the atomizer 500 to have an attractive appearance.

In order to improve an air-tightness of the liquid injection hole 402 sealed by the cover 50, the connecting base 411 is provided with a sealing pad 45 surrounding a periphery of the liquid injection hole 402, for avoiding a e-cigarette liquid leakage.

In the illustrated embodiment, the upper end surface of the connecting base 411. is provided with a sliding rail (not shown), the lower end surface of the covering plate is provided with a sliding block (not shown) engaging the sliding rail. When the sliding member 46 drives the covering plate to slide, the sliding block can slide along the sliding rail, ensuring a sliding stability of the cover 50. It can be understood that, in alternative embodiment not shown, the sliding rail can also be positioned on the covering plate, the sliding block is positioned on the connecting base 411 and engages the sliding rail, a guiding to the sliding of the cover 50 is also provided.

It can be understood that, in alternative embodiment not shown, the sliding member 46 can also be omitted, user can open or close the liquid injection hole 402 by sliding the covering plate.

Further, the atomizer 500 further includes a first locking assembly. It can be understood that, the connecting assembly 40, the cover 50, and the first locking assembly cooperatively constitute a liquid injection protective mechanism. The first locking assembly includes a locking hole positioned on the cover 50, and a locking member extending through the locking hole and detachably connected to the connecting assembly 40. In the illustrated embodiment, the locking hole is a through hole 501 defined on the cover 50, the locking member is a mouth piece 60 extending through the through hole 501 and connected to the connecting assembly 40.

The mouth piece 60 has a substantially hollow cylindrical structure with two openings at opposite ends, after the mouth piece 60 extends through the through hole 501 of the cover 50, the mouth piece 60 is tightly plugged into the connecting base 411. At the same time, the liquid injection hole 402 is sealed by the cover 50, and the mouth piece 60 is fluidly communicating with the aerosol exhaust passage 423, user can aerosol by holding the mouth piece 60 in mouth.

In the illustrated embodiment, when the mouth piece 60 is connected to the connecting assembly 40, the sliding stroke of the cover 50 is restricted by the mouth piece 60, the mouth piece 60 has an outer diameter equal to an inner diameter of the though hole 501 of the cover 50. Specifically, the outer diameter of the portion of the mouth piece 60 matching the through hole 501 is equal to the inner diameter of the through hole 501, because the movable space on the position where the mouth piece 60 matching the through hole 501 is eliminated, therefore, the cover 50 cannot be reversed relative to the mouth piece 60, and the cover 50 is locked. When the mouth piece 60 is dismantled, the cover 50 is then rotatable.

Further, the external wall of the mouth piece 60 is provided with a resisting edge 602, when the mouth piece 60 is connected to the connecting assembly 40, the resisting edge 602 can resist the cover 50, thereby providing a restricting function for the assembly of the mouth piece 60, i.e. when the resisting edge 602 resists the cover 50, it means that the mouth piece 60 is mounted in place. In the illustrated embodiment, the resisting edge 602 is formed by a shrinkage of the neck of the mouth piece 60. It can be understood that, in alternative embodiment not shown, the resisting edge 602 can further be formed by extending outwardly from the circumferential surface of the mouth piece 60 along a radial direction of the mouth piece 60.

The liquid injection protective mechanism further includes a second locking assembly, the second locking assembly includes a first connecting portion and a second connecting portion. It can be understood, the configuration can be referred to that in the third embodiment. The first connecting portion is a latching groove positioned on the connecting base 411. The second connecting portion is a latching member slidably positioned on the cover 50 and corresponding to the latching groove. After the locking member of the first locking assembly is detached from the locking hole, it is required that the latching member and the latching groove to be detached from each other, and thus the liquid injection hole 402 can be opened by a rotation of the cover 50.

In the atomizer 500 provided by the fifth embodiment of the present disclosure, when user want to perform a liquid injection, referring to FIG. 25, the mouth piece 60 is required to be dismantled first, at the time, the locking relationship between the cover 50 and the connecting base 411 of the connecting assembly 40 is removed, and then referring to FIG. 26, the sliding member 46 slides to drive the covering plate to slide, thereby the liquid injection hole 402 is opened. Because the liquid injection hole 402 can only be opened by a complicated operation procedure, thus children cannot open the liquid injection hole causally, thereby a contact and swallow of the e-cigarette liquid for children can be avoided, it is therefore childproof. It can be understood that, referring to a configuration in the first embodiment, a restricting recessed groove 601 is defined on the mouth piece 60 or the cover 50, the other one is provided with a restricting protrusion 5011, by an engagement between the restricting recessed groove 601 and the restricting protrusion 5011, the movable stroke of the mouth piece 601 can be restricted, therefore, the mouth piece 60 cannot lose. When a liquid injection is required, merely the mouth piece 60 is drawn upward to enable the lower end of the mouth piece 60 to be received in the through hole 501.

In the electronic cigarette provided by the fifth embodiment of the present disclosure, all technical features of aforementioned atomizer 500 are included, thus the electronic cigarette has a technical advantage same as that in aforementioned atomizer 500.

### The Sixth Embodiment

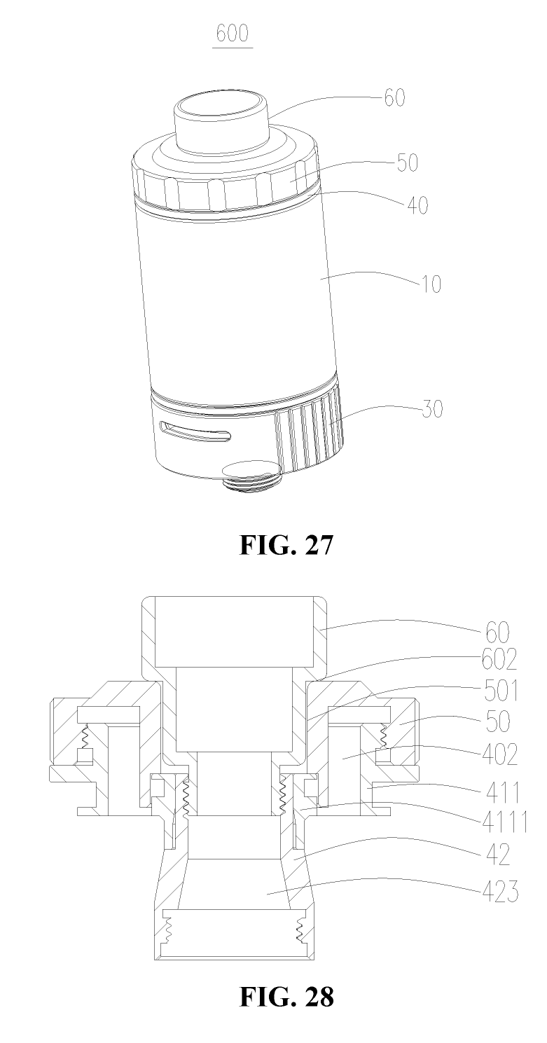

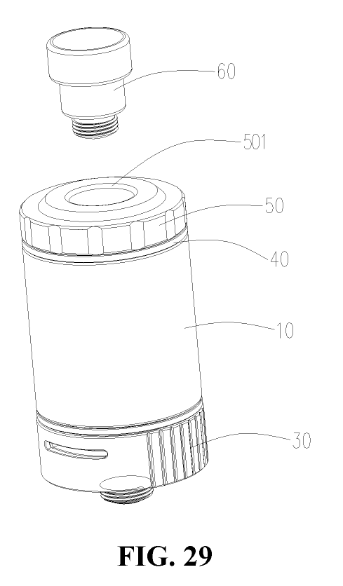

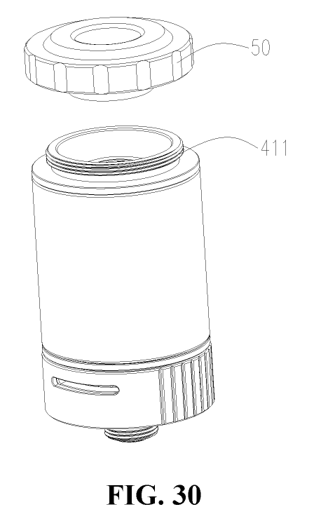

Referring to FIG. 27 and FIG. 30, an electronic cigarette is provided by the sixth embodiment of the present disclosure. The electronic cigarette includes an atomizer 600 and a power source mechanism (not shown) electrically connected to the atomizer 600. Compared to the electronic cigarette of the third embodiment, the difference is that, the structure and the matching mode of the connecting assembly 40 and the cover 50 of the atomizer 600 are different from that in the third embodiment, the housing 10, the atomizing head 20 and the base seat assembly 30 are same as that in the first embodiment, which is not specific described herein.

In the illustrated embodiment, the connecting assembly 40 includes a connecting base 411 connected to an upper end of the housing 10, and a ventilation member 42 connected to the lower end of the connecting base 411. The cover 50 is detachably connected to the connecting base 411, the mouth piece 60 extends through the cover 50 and is connected to the ventilation member 42.

The connecting base 411 is plugged in the upper end of the housing 10, the connecting base 411 has a substantially hollow cylindrical structure with an opening at an upper end. The connecting base 411 is provided with a connecting post 4111 at the center of a bottom of the connecting base 411 for the aerosol to flow out. The lower end of the connecting post 4111 defines two liquid injection holes 402 at opposite sides of the connecting post 4111. The liquid injection holes 402 are fluidly communicating with the liquid storage chamber 101, user can inject e-cigarette liquid into the liquid storage chamber 101 via the liquid injection hole 402. It can be understood that, a number of the liquid injection holes 402 can be one or more than two.

The cover 50 has a substantially hollow cylindrical structure with an opening at a lower end thereof. The cover 50 detachably covers the connecting base 411 and is connected to the connecting base 411 via threads. When the cover 50 covers the connecting base 411, the cover 50 can seal the liquid injection hole 402. In the illustrated embodiment, the cover 50 defines a through hole 501 at a center. It can be understood that, in alternative embodiment not shown, the cover 50 can be detachably connected to the connecting base 411 by latching or plugging.

The ventilation member 42 has a substantially hollow cylindrical structure with two openings at opposite ends. The external wall of the upper end of the ventilation member 42 is connected to the internal wall of the connecting post 4111 by an interference fit. The lower end of the ventilation member 42 is connected to the atomizing head 20 via threads. The interior chamber of the ventilation member 42 forms an aerosol exhaust passage 423, when in use, the aerosol generated in the atomizer 20 enters into user's mouth via the aerosol exhaust passage 423. It can be understood that, in alternative embodiment not shown, the upper end of the ventilation member 42 can be connected to the connecting post 4111 via a threaded connection or latching, the lower end of the ventilation member 42 can be connected to the atomizing head 20 via a threaded connection or latching.

It can be understood that, in alternative embodiment not shown, the upper end of the ventilation member 42 is tightly sleeved on an outer of the connecting post 4111, thus a connection between the ventilation member 42 and the connecting post 4111 can also be established.

Further, the atomizer 400 further includes a first locking assembly. It can be understood that, the connecting assembly 40, the cover 50, and the first locking assembly cooperatively constitute a liquid injection protective mechanism. The first locking assembly includes a locking hole positioned on the cover 50, and a locking member extending through the locking hole and detachably connected to the connecting assembly 40. In the illustrated embodiment, the locking hole is a through hole 501 defined on the cover 50, the locking member is a mouth piece 60 extending through the through hole 501 and connected to the connecting assembly 40.

In the illustrated embodiment, the mouth piece 60 has a substantially hollow cylindrical structure with two openings at opposite ends, after an end of the mouth piece 60 extends through the cover 50, the mouth piece 60 is tightly plugged into the upper end of the ventilation member 42, and the mouth piece 60 is fluidly communicating with the aerosol exhaust passage 423, user can aerosol by holding the mouth piece 60 in mouth. Specifically, after the lower end of the mouth piece 60 extends through the through hole 501 of the cover 50, the lower end of the mouth piece 60 is connected to the ventilation member 42 by a threaded connection. It can be understood that, when the upper end of the ventilation member 42 is tightly sleeved on the outer of the connecting post 4111, the lower end of the mouth piece 60 is connected to the connecting post 4111 via a threaded connection. It can also be understood that, in alternative embodiment not shown, the lower end of the mouth piece 60 can be detachably connected to the ventilation member 42 or the connecting member 4111 by latching or plugging.

Further, the external wall of the mouth piece 60 is provided with a resisting edge 602, when the cover 50 seals the liquid injection hole 402, the resisting edge 602 can resist the cover 50, thereby an axial movement of the cover 50 is restricted, i.e. the cover 50 is locked, a detachment of the cover 50 from the connecting base 411 is avoided. In the illustrated embodiment, the resisting edge 602 is formed by a shrinkage of the neck of the mouth piece 60. It can be understood that, in alternative embodiment not shown, the resisting edge 602 can further be formed by extending outwardly from the circumferential surface of the mouth piece 60 along a radial direction of the mouth piece 60. Further, the resisting edge 602 provides restricting function for the assembly of the mouth piece 60, i.e. when the resisting edge 602 resists the cover 50, it means that the mouth piece 60 is mounted in place.

The liquid injection protective mechanism further includes a second locking assembly, the second locking assembly includes a first connecting portion and a second connecting portion. It can be understood that the configuration can be referred to that in the third embodiment. The first connecting portion is a latching groove positioned on the connecting base 411. The second connecting portion is a latching member slidably poisoned on the cover 50 and corresponding to the latching groove. After the locking member of the first locking assembly is detached from the locking hole, it is required that the latching member to separate from the latching groove, and thus the liquid injection hole 402 can be opened by a rotation of the cover 50.

In the atomizer 600 provided by the sixth embodiment of the present disclosure, when user want to perform a liquid injection, the mouth piece 60 is required to be dismantled first, and then the latching relationship between the cover 50 and the connecting base 411 of the connecting assembly 40 is removed, and then the cover 50 is disassembled from the connecting base 411, the liquid injection hole 402 is thereby opened. Therefore, the liquid injection hole 402 can only be opened by a complicated operation procedure, thus children cannot open the liquid injection hole causally, and a contact and swallow of the e-cigarette liquid for children can be avoided, it is therefore childproof. It can be understood that, referring to a configuration in the first embodiment, a restricting recessed groove 601 is defined on the mouth piece 60 or the cover 50, the other one is provided with a restricting protrusion 5011, by an engagement between the restricting recessed groove 601 and the restricting protrusion 5011, the movable stroke of the mouth piece 601 can be restricted, therefore, the mouth piece 60 cannot lose. When a liquid injection is required, merely the mouth piece 60 is drawn upward to enable the lower end of the mouth piece 60 to be received in the through hole 501.

In the electronic cigarette provided by the sixth embodiment of the present disclosure, all technical features of aforementioned atomizer 600 are included, thus the electronic cigarette has a technical advantage same as that in aforementioned atomizer 600.

It can be understood that, the configuration of the liquid injection protective mechanism in the second embodiment to the sixth embodiment can be referred as that in the first embodiment, a movable ring is added, the movable ring can be movably sleeved on the connecting assembly 40 to avoid a dismantlement of the connecting assembly 40.

The embodiments described above are merely preferred embodiments, but not intended to limit the application. Any modifications, alternatives or improvements made within the principle and spirit of the present application should be interpreted as falling within the protection scope of the present application. The claims are not limited to the features or acts described above. Rather, the proper scope of the disclosure is defined by the appended claims.

What is claimed is:

1. A liquid injection protective mechanism, comprising:
a connecting assembly defining a liquid injection hole;
a cover covering the liquid injection hole and movable relative to the connecting assembly; and
a first locking assembly, comprising a locking hole defined on the cover, and a locking member capable of extending through the locking hole and detachably connected to the connecting assembly;
wherein when an end of the locking member extends through the locking hole and is connected to the connecting assembly, a movable stroke of the cover is restricted by the locking member, for preventing moving the cover to reveal the liquid injection hole.

2. The liquid injection protective mechanism according to claim 1, wherein an outer diameter of a portion of the locking member matching the locking hole is equal to an inner diameter of locking hole, and/or an outer wall of the locking member is provided with a resisting edge, when the end of the locking member extends through the locking hole and is connected to the connecting assembly, the cover is restricted between the resisting edge and the connecting assembly.

3. The liquid injection protective mechanism according to claim 1, wherein a sidewall of the locking hole is provided with a restricting protrusion, an external wall of the locking member defines a restricting recessed groove, or, the sidewall of the locking hole defines a restricting recessed groove, the external wall of the locking member is provided with a restricting protrusion, the restricting protrusion is movably embedded in the restricting recessed groove, for preventing a detachment of the locking member from the cover.

4. The liquid injection protective mechanism according to claim 1, further comprising a movable ring, wherein the movable ring is rotatably sleeved on an outer of the connecting assembly, for preventing the connecting assembly to be dismantled.

5. The liquid injection protective mechanism according to claim 1, wherein the locking member is a mouth piece.

6. The liquid injection protective mechanism according to claim 1, wherein the connecting assembly comprises a connecting base, the cover is detachably connected to the connecting base, or, the cover is movably positioned on the connecting base.

7. The liquid injection protective mechanism according to claim 6, wherein an end of the cover is hinged to the connecting base, allowing the cover to be reversible relative to the connecting base.

8. The liquid injection protective mechanism according to claim 7, wherein a sealing member is protruded from a lower surface of the cover and corresponds to the liquid injection hole, when the cover covers the connecting base, the sealing member is latched into the liquid injection hole.

9. The liquid injection protective mechanism according to claim 6, wherein the cover is rotatably positioned on the connecting base, a rotation center of the cover is staggered from a central axial axis of the connecting base.

10. The liquid injection protective mechanism according to claim 6, wherein the cover is slidably positioned on the connecting base, the connecting base is provided with a sliding rail, and the cover is provided with a sliding block engaging the sliding rail, or, the connecting base is provided with a sliding block, and the cover is provided with a sliding rail engaging the sliding block.

11. The liquid injection protective mechanism according to claim 1, further comprising a second locking assembly, wherein the second locking assembly comprises a first connecting portion positioned on the connecting assembly and a second connecting portion positioned on the cover, after a connection between the locking member and connecting assembly being removed, a movement of the cover is further required to separate the first connecting portion from the second connecting portion, for removing an engagement between the first connecting portion and the second connecting portion, and then the liquid injection hole can be opened.

12. The liquid injection protective mechanism according to claim 11, wherein the connecting assembly defines a translational groove, the cover is provided with a rotation shaft, when the cover is moved, the rotation shaft slides along the translational groove, when the cover reaches a position permitting the first connecting portion to separate from the second connecting member, the cover is capable of rotating around the rotation shaft relative to the connecting assembly to reveal the liquid injection hole.

13. The liquid injection protective mechanism according to claim 12, wherein the connecting assembly comprises a connecting base and a pressing cover covering above the connecting base, the liquid injection hole is defined on the connecting base, the pressing cover defines a cutout with an opening at an end, the cutout is recessed from an edge of a side of the pressing cover, the liquid injection hole is received and revealed in the cutout.

14. The liquid injection protective mechanism according to claim 13, wherein the cutout comprises a connecting wall positioned on a side of the liquid injection hole, and two sidewalls respectively connected to opposite ends of the connecting wall, the two sidewalls extend toward a same direction, a surface of the connecting wall adjacent to the connecting base defines a recessed groove, the recessed groove is recessed from the connecting wall along a direction away from the cutout, the translational groove is defined at an end of each sidewall connecting with the connecting wall, the translation groove is recessed from a surface of the sidewall adjacent to the connecting base, the two surfaces of the two sidewalls defining the translational grooves are opposite to each other, the translational grooves are fluidly communicating with the cutout and the recessed groove.

15. The liquid injection protective mechanism according to claim 14, wherein the cover comprises a movable member, the rotation shaft is positioned on opposite sides of an end of the movable member, a covering portion is formed by extending outwardly from opposite sides of an end of the movable member opposite to the rotation shaft, the pressing cover defines a locking groove on a bottom of an end of the pressing cover away from the connecting wall, the locking groove is fluidly communicating with the cutout, the covering portion forms the second connecting portion, the locking groove forms the first connecting portion, when the movable member is moved to permit the covering portion latching into the locking groove, an end of the movable member adjacent to the rotation shaft is restricted in the recessed groove, when the movable member is moved to enable the covering portion to separate from the locking groove, the end of the movable member adjacent to the rotation shaft is moved out of the recessed groove, the movable member is reversible.

16. The liquid injection protective mechanism according to claim 12, wherein the connecting assembly comprises a lining member and the blocking member, the liquid injection hole is positioned on the lining member, the lining member further defines an embedding groove, the blocking member is embedded in the embedding groove, the translational groove is defined on an end of the blocking member and is arranged along a longitudinal direction of the blocking member, the cover comprises a locking member, the rotation shaft is positioned on an end of the locking member, the connecting assembly further comprises an elastic member, when the locking member is moved, the elastic member resists an end of the locking member provided with the rotation shaft all the time.

17. The liquid injection protective mechanism according to claim 16, wherein the connecting assembly further comprises a snapping member positioned in the embedding groove and beneath an end of the blocking member away from the translational groove, the end of the blocking member away from the translational groove defines a pluggable hole, the snapping member and the pluggable hole cooperative constitute the first connecting portion, the locking member is provided with a claw at an end away from the rotation shaft, the claw forms the second connecting portion, when the locking member is moved to enable the claw to separate from the snapping member, and the claw is aligned to the pluggable hole, the locking member is reversible.

18. The liquid injection protective mechanism according to claim 17, wherein the claw defines a latching groove, the snapping member is provided with a protrusion corresponding to the latching groove, when the first connecting portion engages the second connecting portion, the protrusion latches into the latching groove, or, both the snapping member and the claw are provided with a magnetic member, when the first connecting portion engages the second connecting portion, the snapping member and claw are absorbed by the magnetic members.

19. An atomizer, comprising a liquid injection protective mechanism according to claim 1, further comprising a housing, a base seat assembly, and an atomizing head, wherein the connecting assembly is positioned on an end of the housing, the base seat assembly is positioned on an end of the housing opposite to the connecting assembly, the housing forms a liquid storage chamber fluidly communicating with the liquid injection hole, the atomizing head is received in the liquid storage chamber.

20. An electronic cigarette, comprising an atomizer according to claim 19.
